SEQUENCE LISTING (141 sequences)

SEQ ID NO: 1 - DNA, 20 bp, Artificial
  HOMO SAPIENS
gacaactgactgatactcta

SEQ ID NO: 2 - DNA, 20 bp, Artificial
  HOMO SAPIENS
cgtgtgatgatgatactcta

SEQ ID NO: 3 - RNA, 22 nt, Homo sapiens
ugagguaguagguuguauaguu

SEQ ID NO: 4 - DNA, 42 bp, Artificial
  HOMO SAPIENS
gtcagttgtcaactatacaacctactacctcactccctcttt

SEQ ID NO: 5 - DNA, 42 bp, Artificial
  HOMO SAPIENS
tcatcacacgaactatacaacctactacctcactccctcttt

SEQ ID NO: 6 - DNA, 10 bp, Artificial
  HOMO SAPIENS
aaagagggag

SEQ ID NO: 7 - RNA, 22 nt, Homo sapiens
uagcagcacguaaauauuggcg

SEQ ID NO: 8 - DNA, 42 bp, Artificial
  HOMO SAPIENS
gtcagttgtccgccaatatttacgtgctgctacatcgccttt

SEQ ID NO: 9 - DNA, 42 bp, Artificial
  HOMO SAPIENS
tcatcacacgcgccaatatttacgtgctgctacatcgccttt

SEQ ID NO: 10 - DNA, 10 bp, Artificial
  HOMO SAPIENS
aaaggcgatg

SEQ ID NO: 11 - RNA, 22 nt, Homo sapiens
uagcuuaucagacugauguuga

SEQ ID NO: 12 - DNA, 42 bp, Artificial
  HOMO SAPIENS
gtcagttgtctcaacatcagtctgataagctaccctcgattt

SEQ ID NO: 13 - DNA, 42 bp, Artificial
  HOMO SAPIENS
tcatcacacgtcaacatcagtctgataagctaccctcgattt

SEQ ID NO: 14 - DNA, 10 bp, Artificial
  HOMO SAPIENS
aaatcgaggg

SEQ ID NO: 15 - RNA, 21 nt, Homo sapiens
aucacauugccagggauuacc

SEQ ID NO: 16 - DNA, 41 bp, Artificial
  HOMO SAPIENS
gtcagttgtcggtaatccctggcaatgtgatcggaccattt

SEQ ID NO: 17 - DNA, 41 bp, Artificial
  HOMO SAPIENS
tcatcacacgggtaatccctggcaatgtgatcggaccattt

SEQ ID NO: 18 - DNA, 10 bp, Artificial
  HOMO SAPIENS
aaatggtccg

SEQ ID NO: 19 - RNA, 21 nt, Homo sapiens
uagcaccaucugaaaucgguu

SEQ ID NO: 20 - DNA, 41 bp, Artificial
  HOMO SAPIENS
gtcagttgtcaaccgatttcagatggtgctagccctttgtt

SEQ ID NO: 21 - DNA, 41 bp, Artificial
  HOMO SAPIENS
tcatcacacgaaccgatttcagatggtgctagccctttgtt

SEQ ID NO: 22 - DNA, 10 bp, Artificial
  HOMO SAPIENS
aacaaagggc

SEQ ID NO: 23 - RNA, 23 nt, Homo sapiens
uagcaccauuugaaaucaguguu

SEQ ID NO: 24 - DNA, 43 bp, Artificial
  HOMO SAPIENS
gtcagttgtcaacactgatttcaaatggtgctatcggtgtgtt

SEQ ID NO: 25 - DNA, 43 bp, Artificial
  HOMO SAPIENS
tcatcacacgaacactgatttcaaatggtgctatcggtgtgtt

SEQ ID NO: 26 - DNA, 10 bp, Artificial
  HOMO SAPIENS
aacacaccga

SEQ ID NO: 27 - RNA, 22 nt, Homo sapiens
uguaaacauccuacacucagcu

SEQ ID NO: 28 - DNA, 42 bp, Artificial
  HOMO SAPIENS
gtcagttgtcagctgagtgtaggatgtttacaagtgtcggtt

SEQ ID NO: 29 - DNA, 42 bp, Artificial
  HOMO SAPIENS
tcatcacacgagctgagtgtaggatgtttacaagtgtcggtt

SEQ ID NO: 30 - DNA, 10 bp, Artificial
  HOMO SAPIENS
aaccgacact

SEQ ID NO: 31 - RNA, 21 nt, Homo sapiens
ggcaagaugcuggcauagcug

SEQ ID NO: 32 - DNA, 41 bp, Artificial
  HOMO SAPIENS
gtcagttgtccagctatgccagcatcttgcctaacgcggtt

SEQ ID NO: 33 - DNA, 41 bp, Artificial
  HOMO SAPIENS
tcatcacacgcagctatgccagcatcttgcctaacgcggtt

SEQ ID NO: 34 - DNA, 10 bp, Artificial
  HOMO SAPIENS
aaccgcgtta

SEQ ID NO: 35 - RNA, 23 nt, Homo sapiens
uggcagugucuuagcugguuguu

SEQ ID NO: 36 - DNA, 43 bp, Artificial
  HOMO SAPIENS
gtcagttgtcaacaaccagctaagacactgccagagagaggtt

SEQ ID NO: 37 - DNA, 43 bp, Artificial
  HOMO SAPIENS
tcatcacacgaacaaccagctaagacactgccagagagaggtt

SEQ ID NO: 38 - DNA, 10 bp, Artificial
  HOMO SAPIENS
aacctctctc

SEQ ID NO: 39 - RNA, 23 nt, Homo sapiens
uggagugugacaaugguguuugu

SEQ ID NO: 40 - DNA, 43 bp, Artificial
  HOMO SAPIENS
gtcagttgtcacaaacaccattgtcacactccactctcaggtt

SEQ ID NO: 41 - DNA, 43 bp, Artificial
  HOMO SAPIENS
tcatcacacgacaaacaccattgtcacactccactctcaggtt

SEQ ID NO: 42 - DNA, 10 bp, Artificial
  HOMO SAPIENS
aacctgagag

SEQ ID NO: 43 - RNA, 22 nt, Homo sapiens
uuaaggcacgcggugaaugcca

SEQ ID NO: 44 - DNA, 42 bp, Artificial
  HOMO SAPIENS
gtcagttgtctggcattcaccgcgtgccttaagggtatcgtt

SEQ ID NO: 45 - DNA, 42 bp, Artificial
  HOMO SAPIENS
tcatcacacgtggcattcaccgcgtgccttaagggtatcgtt

SEQ ID NO: 46 - DNA, 20 bp, Artificial
  HOMO SAPIENS
aaaaaaaaaaaacgataccc

SEQ ID NO: 47 - RNA, 23 nt, Homo sapiens
ucccugagacccuuuaaccugug

SEQ ID NO: 48 - DNA, 43 bp, Artificial
  HOMO SAPIENS
gtcagttgtccacaggttaaagggtctcagggatggcctagtt

SEQ ID NO: 49 - DNA, 43 bp, Artificial
  HOMO SAPIENS
tcatcacacgcacaggttaaagggtctcagggatggcctagtt

SEQ ID NO: 50 - DNA, 20 bp, Artificial
  HOMO SAPIENS
aaaaaaaaaaaactaggcca

SEQ ID NO: 51 - RNA, 21 nt, Homo sapiens
cuuuuugcggucugggcuugc

SEQ ID NO: 52 - DNA, 41 bp, Artificial
  HOMO SAPIENS
gtcagttgtcgcaagcccagaccgcaaaaagagggagagtt

SEQ ID NO: 53 - DNA, 41 bp, Artificial
  HOMO SAPIENS
tcatcacacggcaagcccagaccgcaaaaagagggagagtt

SEQ ID NO: 54 - DNA, 20 bp, Artificial
  HOMO SAPIENS
aaaaaaaaaaaactctccct

SEQ ID NO: 55 - RNA, 22 nt, Homo sapiens
cagugcaauguuaaaagggcau

SEQ ID NO: 56 - DNA, 42 bp, Artificial
  HOMO SAPIENS
gtcagttgtcatgcccttttaacattgcactgcctcagtctt

SEQ ID NO: 57 - DNA, 42 bp, Artificial
  HOMO SAPIENS
tcatcacacgatgcccttttaacattgcactgcctcagtctt

SEQ ID NO: 58 - DNA, 20 bp, Artificial
  HOMO SAPIENS
aaaaaaaaaaaagactgagg

SEQ ID NO: 59 - RNA, 22 nt, Homo sapiens
ugagaugaagcacuguagcuca

SEQ ID NO: 60 - DNA, 42 bp, Artificial
  HOMO SAPIENS
gtcagttgtctgagctacagtgcttcatctcagcttgctctt

SEQ ID NO: 61 - DNA, 42 bp, Artificial
  HOMO SAPIENS
tcatcacacgtgagctacagtgcttcatctcagcttgctctt

SEQ ID NO: 62 - DNA, 20 bp, Artificial
  HOMO SAPIENS
aaaaaaaaaaaagagcaagc

SEQ ID NO: 63 - RNA, 22 nt, Homo sapiens
uuaaugcuaaucgugauagggg

SEQ ID NO: 64 - DNA, 42 bp, Artificial
  HOMO SAPIENS
gtcagttgtccccctatcacgattagcattaactgactgctt

SEQ ID NO: 65 - DNA, 42 bp, Artificial
  HOMO SAPIENS
tcatcacacgcccctatcacgattagcattaactgactgctt

SEQ ID NO: 66 - DNA, 20 bp, Artificial
  HOMO SAPIENS
aaaaaaaaaaaagcagtcag

SEQ ID NO: 67 - RNA, 23 nt, Homo sapiens
uauggcacugguagaauucacug

SEQ ID NO: 68 - DNA, 43 bp, Artificial
  HOMO SAPIENS
gtcagttgtccagtgaattctaccagtgccatatgttccgctt

SEQ ID NO: 69 - DNA, 43 bp, Artificial
  HOMO SAPIENS
tcatcacacgcagtgaattctaccagtgccatatgttccgctt

SEQ ID NO: 70 - DNA, 20 bp, Artificial
  HOMO SAPIENS
aaaaaaaaaaaagcggaaca

SEQ ID NO: 71 - RNA, 18 nt, Homo sapiens
uggagagaaaggcaguuc

SEQ ID NO: 72 - DNA, 38 bp, Artificial
  HOMO SAPIENS
gtcagttgtcgaactgcctttctctccagtgcttcctt

SEQ ID NO: 73 - DNA, 38 bp, Artificial
  HOMO SAPIENS
tcatcacacggaactgcctttctctccagtgcttcctt

SEQ ID NO: 74 - DNA, 20 bp, Artificial
  HOMO SAPIENS
aaaaaaaaaaaaggaagcac

SEQ ID NO: 75 - RNA, 21 nt, Homo sapiens
aacuggccuacaaagucccag

SEQ ID NO: 76 - DNA, 41 bp, Artificial
  HOMO SAPIENS
gtcagttgtcctgggactttgtaggccagttggtactcctt

SEQ ID NO: 77 - DNA, 41 bp, Artificial
  HOMO SAPIENS
tcatcacacgctgggactttgtaggccagttggtactcctt

SEQ ID NO: 78 - DNA, 20 bp, Artificial
  HOMO SAPIENS
aaaaaaaaaaaaggagtacc

SEQ ID NO: 79 - RNA, 19 nt, Homo sapiens
gguccagaggggagauagg

SEQ ID NO: 80 - DNA, 39 bp, Artificial
  HOMO SAPIENS
gtcagttgtccctatctcccctctggaccctatggcctt

SEQ ID NO: 81 - DNA, 39 bp, Artificial
  HOMO SAPIENS
tcatcacacgcctatctcccctctggaccctatggcctt

SEQ ID NO: 82 - DNA, 20 bp, Artificial
  HOMO SAPIENS
aaaaaaaaaaaaggccatag

SEQ ID NO: 83 - RNA, 21 nt, Homo sapiens
acagcaggcacagacaggcag

SEQ ID NO: 84 - DNA, 41 bp, Artificial
  HOMO SAPIENS
gtcagttgtcctgcctgtctgtgcctgctgtaccaccactt

SEQ ID NO: 85 - DNA, 41 bp, Artificial
  HOMO SAPIENS
tcatcacacgctgcctgtctgtgcctgctgtaccaccactt

SEQ ID NO: 86 - DNA, 10 bp, Artificial
  HOMO SAPIENS
aagtggtggu

SEQ ID NO: 87 - RNA, 23 nt, Homo sapiens
aaaagcuggguugagagggcgaa

SEQ ID NO: 88 - DNA, 43 bp, Artificial
  HOMO SAPIENS
gtcagttgtcttcgccctctcaacccagcttttcaaccggatt

SEQ ID NO: 89 - DNA, 43 bp, Artificial
  HOMO SAPIENS
tcatcacacgttcgccctctcaacccagcttttcaaccggatt

SEQ ID NO: 90 - DNA, 10 bp, Artificial
  HOMO SAPIENS
aatccggttg

SEQ ID NO: 91 - RNA, 23 nt, Homo sapiens
ugucugcccgcaugccugccucu

SEQ ID NO: 92 - DNA, 43 bp, Artificial
  HOMO SAPIENS
gtcagttgtcagaggcaggcatgcgggcagacacaagggttgt

SEQ ID NO: 93 - DNA, 43 bp, Artificial
  HOMO SAPIENS
tcatcacacgagaggcaggcatgcgggcagacacaagggttgt

SEQ ID NO: 94 - DNA, 20 bp, Artificial
  HOMO SAPIENS
aaaaaaaaaaacaacccttg

SEQ ID NO: 95 - RNA, 21 nt, Homo sapiens
gccugcugggguggaaccugg

SEQ ID NO: 96 - DNA, 41 bp, Artificial
  HOMO SAPIENS
gtcagttgtcccaggttccaccccagcaggcgacaagctgt

SEQ ID NO: 97 - DNA, 41 bp, Artificial
  HOMO SAPIENS
tcatcacacgccaggttccaccccagcaggcgacaagctgt

SEQ ID NO: 98 - DNA, 20 bp, Artificial
  HOMO SAPIENS
aaaaaaaaaaacagcttgtc

SEQ ID NO: 99 - DNA, 15 bp, Artificial
  HOMO SAPIENS
atagtcagtcaacag

SEQ ID NO: 100 - DNA, 15 bp, Artificial
  HOMO SAPIENS
atagtagtagtgtgc

SEQ ID NO: 101 - DNA, 42 bp, Artificial
  HOMO SAPIENS
ccatgttggtcgccaatatttacgtgctgctactgttgactg

SEQ ID NO: 102 - DNA, 42 bp, Artificial
  HOMO SAPIENS
ccatgttggtcgccaatatttacgtgctgctagcacactact

SEQ ID NO: 103 - DNA, 20 bp, Artificial
  HOMO SAPIENS
accaacatggaaaaaaaaaa

SEQ ID NO: 104 - DNA, 42 bp, Artificial
  HOMO SAPIENS
gtgcattggtaactatacaacctactacctcactgttgactg

SEQ ID NO: 105 - DNA, 42 bp, Artificial
  HOMO SAPIENS
gtgcattggtaactatacaacctactacctcagcacactact

SEQ ID NO: 106 - DNA, 20 bp, Artificial
  HOMO SAPIENS
accaatgcactaaaaaaaaa

SEQ ID NO: 107 - DNA, 42 bp, Artificial
  HOMO SAPIENS
cttaggtggttcaacatcagtctgataagctactgttgactg

SEQ ID NO: 108 - DNA, 42 bp, Artificial
  HOMO SEPIENS
cttaggtggttcaacatcagtctgataagctagcacactact

SEQ ID NO: 109 - DNA, 20 bp, Artificial
  HOMO SAPIENS
accacctaaggaaaaaaaaa

SEQ ID NO: 110 - DNA, 41 bp, Artificial
  HOMO SAPIENS
atcatgcggtggtaatccctggcaatgtgatctgttgactg

SEQ ID NO: 111 - DNA, 41 bp, Artificial
  HOMO SAPIENS
atcatgcggtggtaatccctggcaatgtgatgcacactact

SEQ ID NO: 112 - DNA, 20 bp, Artificial
  HOMO SAPIENS
accgcatgatcaaaaaaaaa

SEQ ID NO: 113 - DNA, 41 bp, Artificial
  HOMO SAPIENS
tcgtcatcgtaaccgatttcagatggtgctactgttgactg

SEQ ID NO: 114 - DNA, 41 bp, Artificial
  HOMO SAPIENS
tcgtcatcgtaaccgatttcagatggtgctagcacactact

SEQ ID NO: 115 - DNA, 20 bp, Artificial
  HOMO SAPIENS
acgatgacgaataaaaaaaa

SEQ ID NO: 116 - DNA, 43 bp, Artificial
  HOMO SAPIENS
agtcttgcgtaacactgatttcaaatggtgctactgttgactg

SEQ ID NO: 117 - DNA, 43 bp, Artificial
  HOMO SAPIENS
agtcttgcgtaacactgatttcaaatggtgctagcacactact

SEQ ID NO: 118 - DNA, 20 bp, Artificial
  HOMO SAPIENS
acgcaagactagaaaaaaaa

SEQ ID NO: 119 - DNA, 42 bp, Artificial
  HOMO SAPIENS
ataacggcgtagctgagtgtaggatgtttacactgttgactg

SEQ ID NO: 120 - DNA, 42 bp, Artificial
  HOMO SAPIENS
ataacggcgtagctgagtgtaggatgtttacagcacactact

SEQ ID NO: 121 - DNA, 20 bp, Artificial
  HOMO SAPIENS
acgccgttatacaaaaaaaa

SEQ ID NO: 122 - DNA, 41 bp, Artificial
  HOMO SAPIENS
tatcaggcgtcagctatgccagcatcttgccctgttgactg

SEQ ID NO: 123 - DNA, 41 bp, Artificial
  HOMO SAPIENS
tatcaggcgtcagctatgccagcatcttgccgcacactact

SEQ ID NO: 124 - DNA, 20 bp, Artificial
  HOMO SAPIENS
acgcctgatagtaaaaaaaa

SEQ ID NO: 125 - DNA, 43 bp, Artificial
  HOMO SAPIENS
tcaagtccgtaacaaccagctaagacactgccactgttgactg

SEQ ID NO: 126 - DNA, 43 bp, Artificial
  HOMO SAPIENS
tcaagtccgtaacaaccagctaagacactgccagcacactact

SEQ ID NO: 127 - DNA, 20 bp, Artificial
  HOMO SAPIENS
acggacttgactaaaaaaaa

SEQ ID NO: 128 - DNA, 43 bp, Artificial
  HOMO SAPIENS
ttagagccgtacaaacaccattgtcacactccactgttgactg

SEQ ID NO: 129 - DNA, 43 bp, Artificial
  HOMO SAPIENS
ttagagccgtacaaacaccattgtcacactccagcacactact

SEQ ID NO: 130 - DNA, 20 bp, Artificial
  HOMO SAPIENS
acggctctaatgaaaaaaaa

SEQ ID NO: 131 - DNA, 42 bp, Artificial
  HOMO SAPIENS
ggtaagacgttggcattcaccgcgtgccttaactgttgactg

SEQ ID NO: 132 - DNA, 42 bp, Artificial
  HOMO SAPIENS
ggtaagacgttggcattcaccgcgtgccttaagcacactact

SEQ ID NO: 133 - DNA, 20 bp, Artificial
  HOMO SAPIENS
acgtcttacctcaaaaaaaa

SEQ ID NO: 134 - DNA, 52 bp, Artificial
  HOMO SAPIENS
aacaaacgctacacgtctcgccaatatttacgtgctgctactgttgactgac

SEQ ID NO: 135 - DNA, 20 bp, Artificial
  HOMO SAPIENS
agacgtgtagcgtttgtttc

SEQ ID NO: 136 - DNA, 50 bp, Artificial
  HOMO SAPIENS
ggtttgacgacctagtcgccaatatttacgtgctgctactgttgactgac

SEQ ID NO: 137 - DNA, 20 bp, Artificial
  HOMO SAPIENS
actaggtcgtcaaaccaaga

SEQ ID NO: 138 - DNA, 48 bp, Artificial
  HOMO SAPIENS
ttctgacccttagtcgccaatatttacgtgctgctactgttgactgac

SEQ ID NO: 139 - DNA, 20 bp, Artificial
  HOMO SAPIENS
actaagggtcagaaatgcca

SEQ ID NO: 140 - DNA, 46 bp, Artificial
  HOMO SAPIENS
attcgtcatcgtcgccaatatttacgtgctgctactgttgactgac

SEQ ID NO: 141 - DNA, 42 bp, Artificial
  HOMO SAPIENS
cttaggtggtcgccaatatttacgtgctgctactgttgactg

(19) United States
(12) Patent Application Publication
(10) Pub. No.: US 2009/0061424 A1
(43) Pub. Date: Mar. 5, 2009

(54) UNIVERSAL LIGATION ARRAY FOR ANALYZING GENE EXPRESSION OR GENOMIC VARIATIONS

(75) Inventor: Fuqiang CHEN, St. Louis, MO (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: SIGMA-ALDRICH COMPANY, St. Louis, MO (US)

(21) Appl. No.: 11/847,752

(22) Filed: Aug. 30, 2007

(51) Int. Cl. C12Q 1/68; C40B 40/06

(52) U.S. Cl. 435/6; 506/16

(57) ABSTRACT

The present invention provides an array system comprising a plurality of immobilized oligonucleotides comprising artificial sequences and a plurality of complementary ligation templates, as well as methods and kits for using the array system to analyze populations of nucleic acids. In particular, target nucleic acids are ligated to the immobilized oligonucleotides on the array in the presence of the complementary ligation templates.

### FIELD OF THE INVENTION

The present invention provides an array system, methods, and kits for using the array system to analyze populations of nucleic acids by ligating target nucleic acids to immobilized oligonucleotides on the array.

### BACKGROUND OF THE INVENTION

High throughput parallel assays of gene expression have become increasingly prevalent in drug discovery and many biological fields. Most of these assays are based on nucleic acid hybridization in microarray formats, using glass slides or microbeads as support. While hybridization based techniques may be automated and quantitatively analyzed, they are not well suited for the analysis of genomic variations. For example, hybridization techniques cannot distinguish between target nucleic acids that differ by one nucleotide (i.e., single nucleotide polymorphisms). Thus, there is a need for a high throughput array system that can distinguish between closely related nucleic acids.

Most current array systems comprise organism-specific probes immobilized on the array surface. Furthermore, array systems only exist for organisms whose genomes have been sequenced or that have complete cDNA libraries. Thus, a new array system has to be designed and fabricated each time a different set of targets is to be assayed or an existing set of targets is to be modified or expanded. This not only is time consuming and cost ineffective, but also prohibits the more widespread use of the high throughput technology. “Universal” array systems have been developed in which the arrayed oligonucleotide probes comprise artificial sequences. All of these systems, however, solely rely on nucleic acid hybridization.

In oligonucleotide hybridization, the discriminating power of hybridization sequentially decreases as the position of mismatch moves from the center of the duplex toward the terminus, and therefore hybridization alone often cannot resolve a terminal mismatch. Hybridization alone also cannot distinguish between nucleic acid molecules of different sizes that share complementarity with a particular oligonucleotide probe, but differ in other regions of the molecules. Moreover, in a complex population of nucleic acid molecules, such as a total RNA sample from a mammalian tissue, it is inevitable that some nucleic acid molecules in the population will bear sequences complementary to some of the immobilized artificial oligonucleotides and, as a result, produce unintended hybridization products. Furthermore, hybridization products are not covalently attached to the solid support and, therefore, cannot withstand the most stringent wash conditions that may be necessary for minimizing the array background and maximizing the detection reliability and sensitivity. What is needed, therefore, is a universal array system that provides highly specific sequence discrimination and superior detection reliability and sensitivity, such that closely related microRNAs, single nucleotide polymorphisms, and the like may be analyzed.

### SUMMARY OF THE INVENTION

Among the various aspects of the present invention, therefore, is the provision of a universal array system in which target nucleic acids are ligated to the immobilized oligonucleotides of the array. In particular, the array system comprises a plurality of immobilized oligonucleotides covalently attached to a solid support at a plurality of distinct array positions. Each array position comprises at least one immobilized oligonucleotide comprising a unique artificial sequence. The array system also comprises a plurality of complementary ligation templates. Each ligation template comprises a first region with complementarity to the unique artificial sequence of a specific immobilized oligonucleotide on the array and a second region with complementarity to a specific target nucleic acid. Contact between a particular ligation template with its complementary immobilized oligonucleotide and complementary target nucleic acid directs the target nucleic acid to the immobilized oligonucleotide for subsequent ligation and detection.

Another aspect of the invention encompasses a method for analyzing at least one population of nucleic acids. The method comprises contacting an array of immobilized oligonucleotides with a plurality of target nucleic acids and a plurality of ligation templates. The immobilized oligonucleotides of the array are covalently attached to a solid support at a plurality of distinct array positions. Each array position comprises at least one immobilized oligonucleotide comprising a unique artificial sequence. Each ligation template comprises a first region with complementarity to the unique artificial sequence of a specfic immobilized oligonucleotide and a second region with complementarity to a specific target nucleic acid. Furthermore, each target nucleic acid comprises a signaling means. Upon contact of the array of immobilized oligonucleotides with the plurality of target nucleic acids and the plurality of ligation templates, each target nucleic acid is directed to a specific immobilized oligonucleotide by a specific ligation template. The method further comprises ligating the plurality of target nucleic acids to the plurality of immobilized oligonucleotides in the presence of the plurality of ligation templates, whereby a plurality of ligation products is formed. Each ligation product comprises an immobilized oligonucleotide and a target nucleic acid having a signaling means. The method also comprises quantifying the signal associated with each ligation product, thereby analyzing the population of nucleic acids.

A further aspect of the invention provides a kit for the analysis of at least one population of nucleic acids. The kit comprises an array of immobilized oligonucleotides covalently attached to a solid support at a plurality of distinct array positions, wherein each array position comprises at least one immobilized oligonucleotide comprising a unique artificial sequence. The kit also comprises a plurality of ligation templates, wherein each ligation template comprises a first region with complementarity to the unique artificial sequence of a specific immobilized oligonucleotide and a second region with complementarity to a specific target nucleic acid. Also included in the kit is a template-dependent ligase.

Other aspects and features of the invention are described in more detail herein.

### DESCRIPTION OF THE FIGURES

FIG. 1 diagrams an analysis of mature small RNA molecules using a universal ligation array system. In this embodiment, the immobilized oligonucleotides of the array have free 3′ hydroxyl groups, such that the 5′ end of a mature small RNA molecule is ligated to a specific immobilized oligonucleotide and the 3′ end of the mature small RNA molecule is ligated to a detection tag. The ligations occur in the presence of specific ligation templates, each of which comprises (5′ to 3′) a first region that is complementary to a portion of a detection tag, a second region that is complementary to a mature small RNA, and a third region that is complementary to an immobilized oligonucleotide.

FIG. 2 diagrams another analysis of mature small RNA molecules using a universal ligation array system. In this embodiment, the immobilized oligonucleotides of the array have free 5′ phosphate groups, whereby the 3′ end of a mature small RNA molecule is ligated to a specific immobilized oligonucleotide and the 5′ end of the mature small RNA molecule is ligated to a detection tag. The ligations occur in the presence of specific ligation templates, each of which comprises (5′ to 3′) a first region that is complementary to an immobilized oligonucleotide, a second region that is complementary to a mature small RNA, and a third region that is complementary to a portion of a detection tag.

FIG. 3 diagrams analyses of precursor microRNA molecules (pre-miRNAs) using universal ligation array systems. The pre-miRNAs are labeled by attachment of fluorescent dye molecules. In one embodiment (left), the immobilized oligonucleotides of the array have free 3′ hydroxyl groups, such that the 5′ end of a pre-miRNA molecule is ligated to a specific immobilized oligonucleotide in the presence of a specific ligation template comprising (5′ to 3′) a first region with complementarity to the 5′ end region of the pre-miRNA molecule and a second region with complementarity to the immobilized oligonucleotide. In another embodiment (right), the immobilized oligonucleotides of the array have free 5′ phosphate groups, such that the 3′ end of a pre-miRNA molecule is ligated to a specific immobilized oligonucleotide in the presence of a specific ligation template comprising (5′ to 3′) a first region with complementarity to the immobilized oligonucleotide and a second region with complementarity to the 3′ end region of the pre-miRNA.

FIG. 4 diagrams an analysis of poly(A)⁺ messenger RNA molecules using a universal ligation array. The poly(A) tail of the messenger RNA molecule is removed by digestion with RNase H in the presence of an oligo dT template. The deadenylated messenger RNA fragment is labeled by attachment of fluorescent dye molecules. The 3′ end of the messenger RNA fragment is ligated to the 5′ phosphate group of a specific immobilized oligonucleotide in the presence of a specific ligation template comprising (5′ to 3′) a first region with complementarity to the immobilized oligonucleotide and a second region with complementarity to the 3′ end region of the messenger RNA fragment.

FIG. 5 diagrams an analysis of poly(A)⁻ messenger RNA molecules using a universal ligation array system. The messenger RNA molecule is labeled by attachment of fluorescent dye molecules. The 3′ end of the messenger RNA molecule is ligated to the 5′ phosphate group of a specific immobilized oligonucleotide in the presence of a specific ligation template comprising (5′ to 3′) a first region with complementarity to the immobilized oligonucleotide and a second region with complementarity to the 3′ end region of the messenger RNA molecule.

FIG. 6 diagrams an analysis of messenger RNA molecules using a universal ligation array system. The messenger RNA molecule is digested with RNase H in the presence of a gene-specific DNA template to generate a first RNA fragment with a nascent gene-specific 3′ end and a 3′ hydroxyl group (left) and a second RNA fragment with a nascent gene-specific 5′ end and a 5′ phosphate group (right). The RNA fragments are labeled with fluorescent dye molecules. The 3′ end of the first RNA fragment is ligated to the 5′ phosphate group of a specific immobilized oligonucleotide in the presence of a specific ligation template comprising (5′ to 3′) a first region with complementarity to the immobilized oligonucleotide and a second region with complementarity to the 3′ end region of the first RNA fragment (left). The 5′ end of the second RNA fragments is ligated to the 3′ hydroxyl group of a specific immobilized oligonucleotide in the presence of a specific ligation template comprising (5′ to 3′) a first region with complementarity to the 5′ end region of the second RNA fragment and a second region with complementarity to the immobilized oligonucleotide (right).

FIG. 7 diagrams an analysis of 5′ capped RNA molecules using a universal ligation array system. The RNA molecule is digested with a tobacco acid pyrophosphatase to hydrolyze the phosphoric acid anhydride bonds and generate a 5′ terminal phosphate group on the RNA molecule. The decapped RNA molecule is labeled by attachment of fluorescent dye molecules. The 5′ end of the decapped RNA molecule is ligated to the 3′ hydroxyl group of a specific immobilized oligonucleotide in the presence of a specific ligation template comprising (5′ to 3′) a first region with complementarity to the 5′ end of the decapped RNA molecule and a second region with complementarity to the immobilized oligonucleotide.

FIG. 8 diagrams an analysis of full-length poly(A)⁺ messenger RNA molecules using a universal ligation array system. The full-length poly(A)⁺ messenger RNA is digested with a tobacco acid pyrophosphatase to hydrolyze the phosphoric acid anhydride bonds in the 5′ cap structure and generate a 5′ terminal phosphate group on the full-length poly(A)⁺ messenger RNA molecule. A detection tag comprising at least a fluorescent dye molecule and an oligonucleotide portion is ligated to the 3′ end of the poly(A) tail by a template-dependent ligase and facilitated by a generic ligation template. The generic ligation template comprises (5′ to 3′) a first region that is complementary to the oligonucleotide portion of the detection tag and a second region that is complementary to the 3′ end region of the poly(A) tail. The 5′ end of the decapped and labeled full-length poly(A)⁺ messenger RNA molecule is ligated to the 3′ hydroxyl group of a specific immobilized oligonucleotide in the presence of a specific ligation template comprising (5′ to 3′) a first region with complementarity to the 5′ end of the decapped RNA molecule and a second region with complementarity to the immobilized oligonucleotide.

FIG. 9 diagrams an analysis of DNA molecules using a universal ligation array system. A target DNA molecule corresponding to a region of interest in a DNA molecule is generated by primer extension and ligation, PCR amplification and labeling, and endonuclease and exonuclease digestions. The 3′ end of the resultant single stranded target DNA molecule is ligated to the 5′ phosphate group of a specific immobilized oligonucleotide in the presence of a specific ligation template that comprises (5′ to 3′) a region with complementarity the immobilized oligonucleotide and a region with complementarity to the 3′ end region of the target DNA molecule.

### DETAILED DESCRIPTION OF THE INVENTION

It has been discovered that target nucleic acids may be ligated to a universal array of immobilized oligonucleotides by a template-dependent ligase in the presence of complementary ligation templates. Each ligation template has a first region with complementarity to the unique artificial sequence of a specific immobilized oligonucleotide and a second region with complementarity to a specific target nucleic acid, whereby the ligation template facilitates ligation between the target nucleic acid and the immobilized oligonucleotide. The resultant ligation products, therefore, are covalently attached to the array, and all non-covalently attached molecules may be removed by exposure to stringent wash conditions. The ligation array system and its methods of use not only provide stringent sequence discrimination, but also size and functional group discrimination (e.g., a mature microRNA may be distinguished from its precursor microRNA on the basis of size and its unique terminal sequence). Furthermore, the array of this invention provides the flexibility of a universal array system, i.e., the same array may be used for analyzing populations of target nucleic acid molecules from virtually any organism, and new target nucleic acids may be readily analyzed by designing and making new sets of ligation templates, rather than fabricating new arrays.

### (I) Ligation Array System

One aspect of the present invention provides a ligation array system for analyzing nucleic acids. The array system comprises a plurality of arrayed immobilized oligonucleotides and a plurality of ligation templates. The immobilized oligonucleotides of the array are covalently attached to a solid support at a plurality of distinct array positions, whereby each array position comprises at least one immobilized oligonucleotide comprising a unique artificial sequence. Each ligation template comprises a first region with complementarity to the unique artificial sequence of a specific immobilized oligonucleotide and a second region with complementarity to all or part of a specific target nucleic acid. Each ligation template, therefore, is capable of directing a specific target nucleic acid to a specific immobilized oligonucleotide and facilitating the ligation between the immobilized oligonucleotide and the target nucleic acid. Each ligation template may also comprise a third region with complementarity to a portion of a detection tag, such that it may facilitate the ligation between the detection tag and the target nucleic acid.

The array system of the invention differs from most other arrays in that the target nucleic acid is ligated to an immobilized oligonucleotide on the array. Thus, ligated products rather than hybridized products may be detected. Because the sequences of the immobilized oligonucleotides on the array are artificial and require no complementarity to the sequences of any organism for target detection, the array is universal and may be used to analyze any population of nucleic acids from any organism. The ligation templates provide the sequence specificity for a particular population of nucleic acids.
(a) Immobilized Oligonucleotides Covalently Attached to a Solid Support

The array system comprises a plurality of immobilized oligonucleotides covalently attached to a solid support via their 5′ or 3′ ends at a plurality of array positions. Each immobilized oligonucleotide comprises a unique artificial sequence.

(i) oligonucleotides

The array comprises a plurality of immobilized oligonucleotides covalently attached to a solid support. The immobilized oligonucleotides of the invention are single stranded molecules. The immobilized oligonucleotides may be deoxyribonucleic acids, ribonucleic acids, or combinations thereof. The lengths of the immobilized oligonucleotides can and will vary, depending on the application. For example, the immobilized oligonucleotide may range from about 4 nucleotides to several hundred nucleotides in length. Regardless of its length, however, each immobilized oligonucleotide comprises a unique artificial sequence at its free end. Each unique artificial sequence may range from about 4 nucleotides to about 30 nucleotides in length. In preferred embodiments, each unique artificial sequence may be 6, 7, 8, 9, 10, 11, 12, 13, 14, 15, 16, 17, 18, 19, 20, 21, 22, 23, or 24 nucleotides in length. In an exemplary embodiment, the unique artificial sequence of a plurality of immobilized oligonucleotides may be about 14-18 nucleotides in length.

The unique sequences of the immobilized oligonucleotides are artificial, i.e., the sequences are randomly generated with no intended complementarity to those of any known organism. The artificial sequences may be selected by a computer program from a pool of random combinations of the fours nucleotides, A, C, G, and T/U. As an example, a set of artificial sequences comprising 12 nucleotides may be selected from more than 16 million different combinations. Typically, the sequences will be selected such that they are sufficiently different from one another to prevent cross-hybridization. Furthermore, the sequences that are selected will be devoid of self-complementarity (i.e., secondary structure). In general, the artificial sequences comprising an array will have similar thermodynamic properties (e.g., have similar percentages of G and C or similar melting temperatures) such that hybridization with the plurality of ligation templates may be performed simultaneously under one reaction condition.

The number of distinct immobilized oligonucleotides comprising an array can and will vary, depending upon the application and the solid support. In general, the number of distinct immobilized oligonucleotides comprising an array may range from about two immobilized oligonucleotides to millions of immobilized oligonucleotides. In some embodiments, the number of distinct immobilized oligonucleotides may range from about 1,000,000 distinct immobilized oligonucleotides to about 100,000 distinct immobilized oligonucleotides, from about 100,000 distinct immobilized oligonucleotides to about 10,000 distinct immobilized oligonucleotides, from about 10,000 distinct immobilized oligonucleotides to about 1,000 distinct immobilized oligonucleotides, from about 1,000 distinct immobilized oligonucleotides to about 100 distinct immobilized oligonucleotides, or from about 100 distinct immobilized oligonucleotides to about 2 distinct immobilized oligonucleotides. In a preferred embodiment, the array may comprise about 4,000 to 10,000 distinct immobilized oligonucleotides.

(ii) solid support

Each of the distinct oligonucleotides is covalently attached to a solid support. The solid support may be made of any material that is amenable to covalent attachment of the oligonucleotides. In general, useful solid support materials include those that are substantially transparent to visible and/or UV light. The solid support material may be flexible or rigid. Non-limiting examples of materials include glass; modified glass; functionalized glass; silica; silica-based materials; silicon; structured silicon; modified silicon; polymers, such as polysaccharides, celluloses, acrylics, polystyrenes, polypropylene, polyethylene, polybutylene, polyurethane, polycarbonate, polytetrafluoroethylene, and so forth; copolymers; metals, such as gold, platinum, titanium, and the like; and membranes, such as nylon, modified nylon, nitrocellulose, modified nitrocellulose, and so forth.

The surface of the solid support may be further modified to comprise a thin layer of three-dimensional porous structures. In a preferred embodiment, the surface of the solid support may be further modified to comprise a thin layer of a hydrophilic polymer gel, such as that of CodeLink slides (available from Amersham Biosciences, Piscataway, N.J.). In another embodiment, the surface of the solid support may be further modified to comprise a thin layer of dendrimers. For example, the surface may comprise a thin layer of cross-linked polyamidoamine (PAMAM) starburst dendrimers

The form or shape of the solid support may vary, depending on the application. Suitable examples include, but are not limited to, slides, strips, plates, wells, microparticles, fibers (such as optical fibers), gels, and combinations thereof. Slides may have rectangular, square, or circular shapes, and the dimensions of the slide may vary. Plates may be microtiter plates with 96, 384, or 1536 wells. The microtiter plates may be further modified to have bead wells in the bottom of the assay wells. Microparticles may be spherical or they may have irregular shapes. The size of the microparticles may range from about 100 nanometers to about 1 millimeter, with microparticles of about 0.5 micron to about 5 microns, about 5 microns to about 50 microns, or about 50 microns to about 200 microns being particularly useful.

In one embodiment, the solid support may be a glass microscope slide. In another embodiment, the solid support may comprise microparticles immobilized on a microtiter plate. In still another embodiment, the solid support may be microparticles embedded in etched optical fiber bundles that are assembled into a matrix that matches a microtiter plate. In a further embodiment, the solid support may be microparticles that are internally color-coded with unique combinations of spectrally distinct fluorescent dyes.

Additionally, the solid support comprises a plurality of individual array positions that are physically separated from each other, such that distinct immobilized oligonucleotides may be attached to the solid support at distinct array positions. The physical separation of the array positions may be due to the presence of wells; depressions; etched trenches; raised regions; physical barriers, such as a removable seal or gasket; or chemical barriers, such as hydrophobic or hydrophilic regions that repel the flow of aqueous or nonpolar solvents, respectively. The array positions may also be introduced on the surface of the solid support by a variety of techniques including, but not limited to, photolithography, stamping techniques, molding techniques, printing techniques, and microetching techniques. In general, the array will have a regular pattern such that each array position may be assigned a unique address. In some embodiments, the address may be planar, e.g., may be defined in terms of the X and Y coordinates. Accordingly, immobilized oligonucleotides attached to microtiter plates or slides may have planar addresses. In other embodiments, such as those comprising microparticles, the address may be spectral, a unique nucleic acid sequence, or a combination thereof.

(iii) covalent attachment

The immobilized oligonucleotides are covalently attached to a solid support. The covalent linkage may be formed by reacting a functional group on the surface of the solid support with a functional group on an oligonucleotide. Non-limiting examples of suitable functional groups that may be used include N-hydroxysuccinimide (NHS) ester, epoxy, acyl halide, aldehyde, amino, carboxyl, chloromethyl, halo, hydroxyl, keto, silanol, and sulfonate.

In some embodiments, a functional group may preexist on the surface of the solid support. For example, silica-based materials have silanol groups, polysaccharides have hydroxyl groups, and synthetic polymers may have a broad range of reactive groups, depending upon the monomers from which they are constructed. Alternatively, suitably modified solid supports may be obtained from any of several commercial suppliers. In other embodiments, the solid support may be further modified, reacted, or coated to introduce a functional group, using techniques known to those of skill in the art. For example, amino groups may be added to glass- or silica-based solid supports by reaction with an amine compound such as 3-amino-propyl triethoxysilane, 3-aminopropylmethyldiethoxysilane, 3-aminopropyl dimethylethoxysilane, and the like. Other suitable treatments include chromic acid oxidation, plasma amination, or reaction with a functionalized side chain alkyltrichlorosilane.

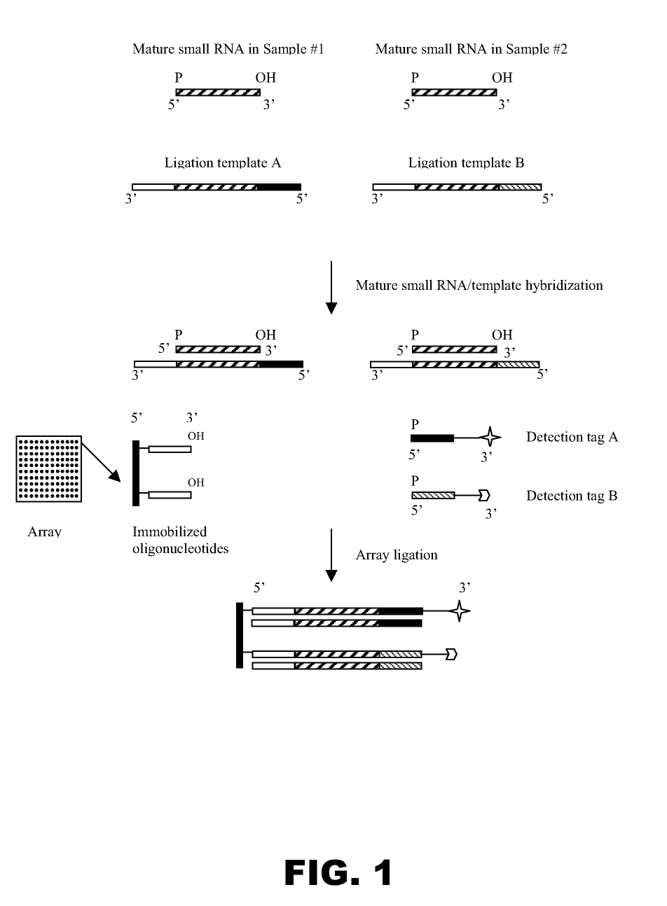

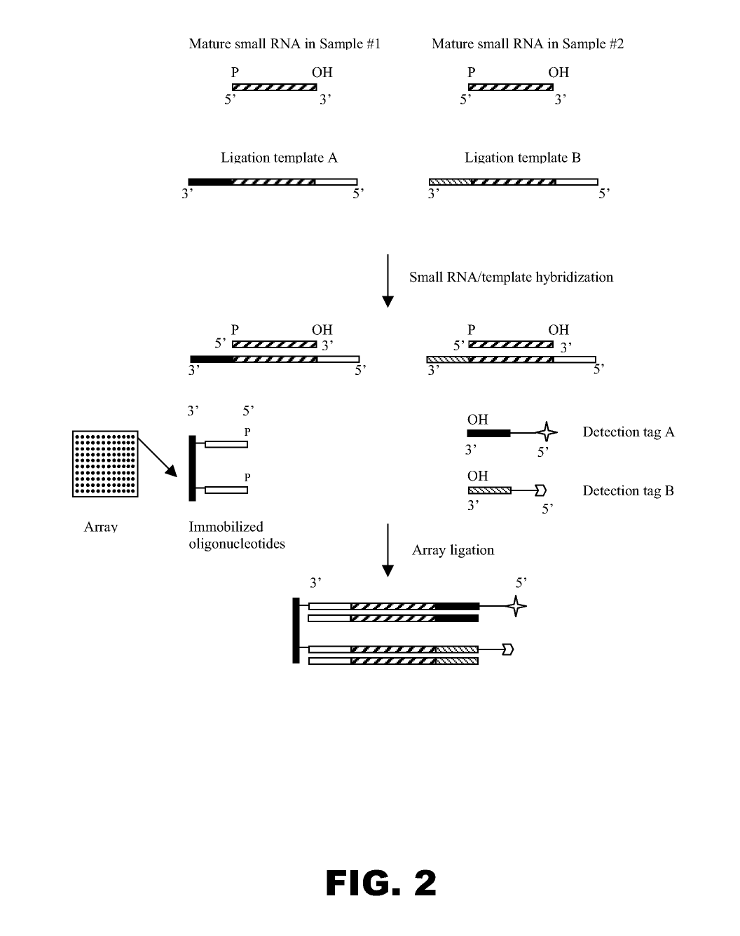

Similarly, the oligonucleotide may be modified such that it contains a reactive functional group. For example, an amino group may be added to the oligonucleotide. The functional group may be positioned at the 5′ end of the oligonucleotide, whereby the 5′ end of the oligonucleotide may be covalently linked to the solid support and the 3′ end of the oligonucleotide remaining free for subsequent ligation (e.g., see FIG. 1). Alternatively, the functional group may be located at the 3′ end of the oligonucleotide, such that the 3′ end of the oligonucleotide may be covalently linked to the solid support and the 5′ end of the oligonucleotide being free for subsequent ligation (e.g., see FIG. 2). In embodiments in which the 3′ end of the oligonucleotide is linked to the solid support, the 5′ end of the oligonucleotide will generally contain a 5′ terminal phosphate group for the subsequent ligation reaction. The 5′ terminal phosphate group may be introduced during the synthesis of the oligonucleotide. Alternatively, 5′ terminal phosphate group may be added in situ after the oligonucleotide has been coupled to the solid support. In one embodiment, the phosphate group may be added by enzymatic phosphorylation with a polynucleotide kinase. The polynucleotide kinase may be from T4 bacteriophage or a thermophilic bacteriophage. In another embodiment, the phosphate group may be added via chemical phosphorylation (e.g., Horn and Urdea (1986) Tetrahedron Lett. 27(39):4705-4708).

The type of covalent coupling reaction can and will vary, depending upon the functional groups on the oligonucleotide and the solid support. Those of skill in the art will be familiar with techniques to accomplish the appropriate coupling chemistry. For example, amino groups may be covalently attached to a solid support comprising N-hydroxysuccinimide ester or epoxy functional groups in a humidity chamber, hydroxyl groups may be incorporated into stable carbamate linkages by several methods; amino groups may be acylated directly, and carboxyl groups may be activated by reaction with N,N′-carbonyldiimidazole or water-soluble carbodiimides and then reacted with an amino group.

In some embodiments, a linker may be disposed between the immobilized oligonucleotide and the solid support. The linker will generally be of sufficient length and flexibility to permit ready interaction between the immobilized oligonucleotides and the ligation templates. In general, the linker may range from about 5 atoms to about 50 atoms in length. The hydrophilic/hydrophobic properties and the charge of the linker can and will vary, depending upon the embodiment. The chain of atoms defining the linker will typically be selected from the group consisting of carbon, oxygen, nitrogen, sulfur, selenium, silicon and phosphorous. In some embodiments, the linker may be a hydrocarbyl or a substituted hydrocarbyl chain. The hydrocarbyl chain may be saturated, unsaturated, linear, cyclic, or branched. In general, the linker comprises at least two functional groups—a first to react with the solid support and a second to react with the oligonucleotide—such that the linker is disposed between the solid support and the immobilized oligonucleotide. Types of functional groups and types of linkages were discussed above. The linker may be uniformly attached to the solid support, or the linker may be attached to the solid support in an ordered array.

The oligonucleotides are immobilized at specific positions on the surface of the solid support of the array. In some embodiments, the immobilized oligonucleotides may be synthesized directly on the solid support using nucleic acid synthesis techniques well known in the art. In other embodiments, the oligonucleotides may be deposited onto the solid support using a variety of printing, lithographic, and deposition techniques known to those of skill in the art. Each array position comprises at least one immobilized oligonucleotide comprising a unique artificial sequence. In one embodiment, each array position may comprise one distinct immobilized oligonucleotide. In another embodiment, each array position may comprise two distinct immobilized oligonucleotides. In yet another embodiment, each array position may comprise more than two distinct immobilized oligonucleotides. Furthermore, an array position may contain more than one copy of each distinct oligonucleotide. In general, the amount of a distinct oligonucleotide present at an array position will be at least 0.1 zeptomole. While the number of array positions may vary among the arrays, the configuration of the arrays may also vary. For example, an array may comprise more than one array position in which identical oligonucleotides are attached, such that the array comprises duplicates of that distinct oligonucleotide(s). Stated another way, an array may comprise subsets of smaller arrays. Furthermore, an array may comprise some positions in which the oligonucleotides are attached to the solid support by their 5′ ends with their 3′ ends free for ligation, and other positions in which the oligonucleotides are attached by their 3′ ends with their 5′ ends free for ligation.
(b) Ligation Templates

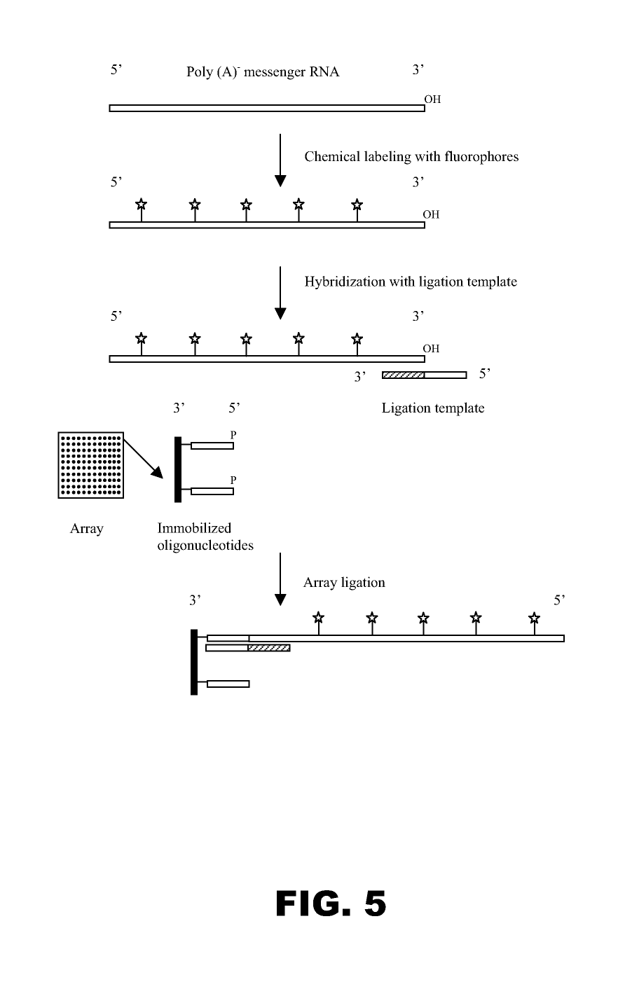

The array system also comprises a plurality of ligation templates. The ligation templates are single-stranded oligonucleotides. Accordingly, they may be deoxyribonucleic acids, ribonucleic acids, or combinations thereof. Each ligation template comprises a first region that is complementary to the unique artificial sequence of a specific immobilized oligonucleotide on the array and a second region that is complementary to all or a part of a specific target nucleic acid. A ligation template may further comprise a third region that is complementary to a portion of a detection tag. Detection tags are described below in section (II)(a)(i). The configuration or orientation of a ligation template may vary: the region with complementarity to the unique artificial sequence of an immobilized oligonucleotide may be at the 5′ end or the 3′ end of a ligation template; the region with complementarity to the target nucleic acid may be at the 5′ end, the 3′ end or the middle of a ligation template; and the optional region with complementarity to a portion of a detection tag may be at the 5′ end or the 3′ end of the ligation template (e.g., see FIGS. 1-9).

In some embodiments, a ligation template may comprise a pair of oligonucleotides. The pair comprises a first oligonucleotide having a first region with complementarity to the unique artificial sequence of an immobilized oligonucleotide and a second region with complementarity to a first portion of a target nucleic acid, and a second oligonucleotide comprising a first region with complementarity to a second portion of the same target nucleic acid and a second region with complementarity to a portion of a detection tag. Hybridization between a ligation template and a target nucleic acid and an immobilized oligonucleotide guides the target nucleic acid to a particular immobilized oligonucleotide at a specific array position.

The length of the ligation templates can and will vary, depending mainly upon the type of nucleic acid to be analyzed. Similarly, the length of each separate region of a ligation template may vary. That is, the region with complementarity to the unique artificial sequence of an immobilized oligonucleotide may range from about 4 nucleotides to about 30 nucleotides in length; the region with complementarity to a target nucleic acid may range from about 8 nucleotides to about 60 nucleotides in length; and the optional region with complementarity to a detection tag may range from about 6 nucleotides to about 20 nucleotides in length.

The number of ligation templates comprising an array can and will vary depending upon the application. In some embodiments, the number of distinct ligation templates may be less than the number of distinct immobilized oligonucleotides comprising the array, i.e., some immobilized oligonucleotides are left vacant for future expansion of target nucleic acids. In a preferred embodiment, the number of distinct ligation templates will generally be at least equal to the number of distinct immobilized oligonucleotides comprising the array. Furthermore, depending upon the signaling means used to detect the target nucleic acid, additional ligation templates may be used (e.g., see Examples 1 and 2).

The amount of each ligation template comprising an array can and will vary depending upon the number of oligonucleotides immobilized on an array and the type of target nucleic acid being analyzed. The amount of each ligation template may range from about 0.5 attomoles (amoles) to about 500 femtomoles (fmoles). For example, the amount of each ligation template may range from about 0.5 amoles to about 5 amoles, from about 5 amoles to about 50 amoles, from about 50 amoles to about 500 amoles, from about 0.5 fmoles to about 5 fmoles, from about 5 fmoles to about 50 fmoles, or from about 50 fmoles to about 500 fmoles.

A ligation template may be used to distinguish between closely related target nucleic acids. As an example, many microRNAs are generated via the cleavage of much larger precursor microRNAs. Although a ligation template having complementarity to a microRNA may bind to a portion of its larger precursor, the ligation template cannot facilitate the ligation of the precursor to the immobilized oligonucleotide designated for the mature microRNA because of the additional unhybridizable sequence of the large precursor molecule.

In addition to guiding a target nucleic acid to specific array position, a ligation template also facilitates ligation between the target nucleic acid and the immobilized oligonucleotide at that array position. Thus, hybridization between a ligation template and an immobilized oligonucleotide and a target nucleic acid positions the 3′ terminal hydroxyl group of one in close proximity of the 5′ terminal phosphate group of the other such that a phosphodiester bond may be formed by a template-dependent ligase, thereby ligating the target nucleic acid to the immobilized oligonucleotide. Thus, ligation templates may be designed such that a specific target nucleic acid may be directed to and ligated with a particular immobilized oligonucleotide in a particular orientation at a specific array position.

Each ligation template may further comprise at least one locked nucleic acid (LNA). In general, the inclusion of a LNA increases the melting temperature of the ligation template and, consequently, may be used to increase the specificity of hybridization. Without being bound by any particular theory, LNAs may be used to help discriminate between closely related target nucleic acids (e.g., microRNAs that differ by one or two nucleotides). Furthermore, the ligation templates may also comprise non-nucleic acid molecules, such as biotin or digoxigenin. For example, biotin-modified ligation templates may be used to capture and concentrate the target nucleic acids from crude cell lysates and/or extremely diluted samples.

### (II) Method for Analyzing at Least One Population of Nucleic Acids

Another aspect of the invention provides a method for analyzing at least one population of nucleic acids using the ligation array system of the invention. The method comprises ligating specific target nucleic acids to the immobilized oligonucleotides on the array and detecting the ligated products at the distinct array positions. Detecting and analyzing ligated products, rather than hybridized products, increases the specificity of detection.
(a) Contacting an Array of Immobilized Oligonucleotides with a Plurality of Target Nucleic Acids in the Presence of a Plurality of Ligation Templates

The method comprises contacting an array of immobilized oligonucleotides with a plurality of target nucleic acids and a plurality of ligation templates. The universal array of immobilized oligonucleotides covalently attached to a solid support was detailed above in section (I)(a). Each immobilized oligonucleotide comprises a unique artificial sequence. The ligation templates were described above in section (I)(b). Each ligation template comprises a first region that is complementary to the unique artificial sequence of a specific immobilized oligonucleotides and a second region that is complementary to all or part of a specific target nucleic acid. Each ligation template may also comprise an optional third region that is complementary to a portion of a detection tag.

(i) target nucleic acid

The type of target nucleic acid that is contacted with and ligated to the array of the invention can and will vary. The target nucleic acids may be RNA molecules, DNA molecules, or combinations thereof. In some embodiments, the target nucleic acids that are contacted with the array may be the population of nucleic acids that is being analyzed. For example, when a population of small RNA molecules is being analyzed, the target nucleic acids contacted with the immobilized oligonucleotides of an array will generally be the small RNA molecules themselves. In other embodiments, the target nucleic acids that are contacted with an array may be fragments of messenger RNA molecules or genomic DNA molecules. For example, the fragments may be generated by enzyme digestion (e.g., restriction endonuclease digestion of double-stranded DNA or RNase H digestion of an RNA/DNA hybrid). Alternatively, the fragments may be generated by the physical shearing of double-stranded DNA or single-stranded RNA. In other embodiments, the target nucleic acid may be derived from the population of nucleic acids that is being analyzed. For example, the target nucleic acids that are contacted with the array may be cDNA or cRNA copies of messenger RNA molecules or fragments thereof. Similarly, the target nucleic acids may be PCR-amplified copies of genomic DNA molecules or fragments thereof. In still other embodiments, the target nucleic acids contacted with an array may be chemically synthesized nucleic acids, or the target nucleic acid may be a combination of a naturally occurring nucleic acid and a synthetic nucleic acid.

Non-limiting examples of populations of nucleic acids that may be used as target nucleic acids or may be used to generate target nucleic acids include mature microRNA (miRNA), mature short interfering RNA (siRNA), mature repeat-associated siRNA (rasiRNA), mature transacting siRNA (tasiRNA), mature Piwi-interacting RNA (piRNA), mature 21U-RNA, precursor small RNA, precursor microRNA (pre-miRNA), small nuclear RNA (snRNA), small nucleolar RNA (snoRNA), messenger RNA (mRNA), 23S/28S (or 16S/18S) ribosomal RNA (rRNA), 5.8S rRNA, 5S rRNA, transfer RNA (tRNA), genomic DNA, and organellar DNA. The population of nucleic acids may be derived from eukaryotes, eubacteria, archaea, or viruses. Non-limiting examples of suitable eukaryotes include humans, mice, mammals, vertebrates, invertebrates, plants, fungi, yeast, and protozoa. The nucleic acids may be derived from a cell, a cell extract, a tissue from a multicellular organism, a whole organism, a body fluid, or any other nucleic acid-containing preparation (e.g., a synthetic preparation). Non-limiting examples of a suitable body fluid include blood, serum, saliva, cerebrospinal fluid, pleural fluid, lymphatic fluid, milk, sputum, semen, and urine.

The length of the target nucleic acid contacted with an array can and will vary, depending upon the type of nucleic acid being analyzed. In an embodiment in which a population of mature miRNAs (or siRNAs) is being analyzed, the target nucleic acids that are contacted with an array may be the mature miRNAs (or siRNAs), which range from about 16 nucleotides to about 23 nucleotides in length. In another embodiment in which a population of mature piRNAs is being analyzed, the target nucleic acids may be the mature piRNAs, which range from about 26 nucleotides to about 31 nucleotides in length. In still another embodiment, a population of precursor microRNAs may be analyzed and the target nucleic acids may be the precursor microRNAs, which range from about 60 nucleotides to about 160 nucleotides in length. In yet another embodiment, a population of messenger RNA molecules may be being analyzed, the target nucleic acids that are contacted with the array may be the messenger RNA molecules, which may range from about 100 nucleotides to about 10,000 nucleotides in length. Alternatively, the target nucleic acids that are contacted with the array may be the fragments of the messenger RNA molecules, and the RNA fragments may range from about 100 nucleotides to about 5,000 nucleotides in length. In yet another embodiment, regions of interest in messenger RNA molecules or genomic DNA molecules may be analyzed, the target nucleic acids that are contacted with the array may be cDNA copies or amplified copies of the regions of interest, and these target nucleic acids may range from about 50 nucleotides to about 500 nucleotides in length.

The amount of target nucleic acid contacted with an array can and will vary, depending upon the type of nucleic acid being analyzed and the purity of the target nucleic acid preparation. The amount of target nucleic acid may range from about 1 ng to about 20 μg. In one embodiment, the amount of target nucleic acid may range from about 1 ng to about 30 ng. In another embodiment, the amount of target nucleic acid may range from about 30 ng to about 100 ng. In an alternate embodiment, the amount of target nucleic acid may range from about 100 ng to about 300 ng. In yet another embodiment, the amount of target nucleic acid may range from about 300 ng to about 1000 ng. In still another embodiment, the amount of target nucleic acid may range from about 1 μg to about 10 μg. In another embodiment, the amount of target nucleic acid may range from about 10 μg to about 20 μg.

The target nucleic acids that are contacted with the immobilized oligonucleotides in the presence of the ligation templates are generally single-stranded molecules. Thus, single-stranded target nucleic acids may hybridize with the ligation templates, and single-stranded target nucleic acids may be ligated to the single-stranded oligonucleotides immobilized on the array. In embodiments in which the starting target nucleic acid or a portion thereof is double-stranded, the target nucleic acid will generally be made single-stranded prior to contact with the ligation templates and the arrayed immobilized oligonucleotides. A double-stranded nucleic acid may be converted to a single-stranded nucleic acid by heating from about 75° C. to about 100° C.

Furthermore, if a target nucleic acid is to be ligated via its 5′ end to a specific immobilized oligonucleotide (or a detection tag, as described below), typically the 5′ end will comprise a terminal phosphate group. The 5′ terminal phosphate may be naturally occurring, it may be part of a primer used during an amplification step, or it may be added enzymatically with a polynucleotide kinase. The polynucleotide kinase may be from T4 bacteriophage or a thermophilic bacteriophage.

(ii) signaling means

The target nucleic acids that are contacted with and ligated to the immobilized oligonucleotides of an array also comprise signaling means. The signaling means generates a signal such that the ligated target nucleic acids may be detected and quantified. The signaling means may comprise at least one signaling molecule covalently attached to the target nucleic acid, or the signaling means may comprise a detection tag, comprising at least one signaling molecule, that is ligated to the target nucleic acid.

A signaling molecule may be a fluorescent dye (fluorophore), such as fluorescein and its derivatives such as FAM, HEX, TET, and TRITC; rhodamine and its derivatives such as ROX and Texas Red; R-phycoerythin; the Cy dyes such as Cy3 and Cy5 (Amersham Biosciences); and the Alexa fluor dyes (Molecular Probes/Invitrogen, Carlsbad, Calif.). In another embodiment, a signaling molecule may be a molecule such as biotin or digoxigenin whose detection is indirect, i.e., comprises additional reagents and/or manipulations prior to detection. In still another embodiment, the signaling molecule may comprise a modified nucleotide, e.g., aminoallyl-dUTP or bromo-dUTP, which may be detected indirectly. In yet a further embodiment, the signaling molecule may comprise a sequence of nucleotides that is a target for branched DNA (bDNA) detection. Briefly, one end of the bDNA molecule is designed to bind the sequence of nucleotides in the target nucleic acid, while the other end of the bDNA molecule contains many branches of DNA that are designed to bind a probe used for signal detection. In another alternate embodiment, the signaling molecule may comprise nanocrystals or quantum dots, such as CdSe nanocrystals, III-nitride quantum dots, and EVIFLUOR® quantum dots (Evident Technologies, Troy, N.Y.). In still other embodiments, the signaling molecule may comprise magnetic probes, heavy metals, phosphorescent groups, radioactive moieties, chemiluminescent moieties, or electrochemical detecting moieties.

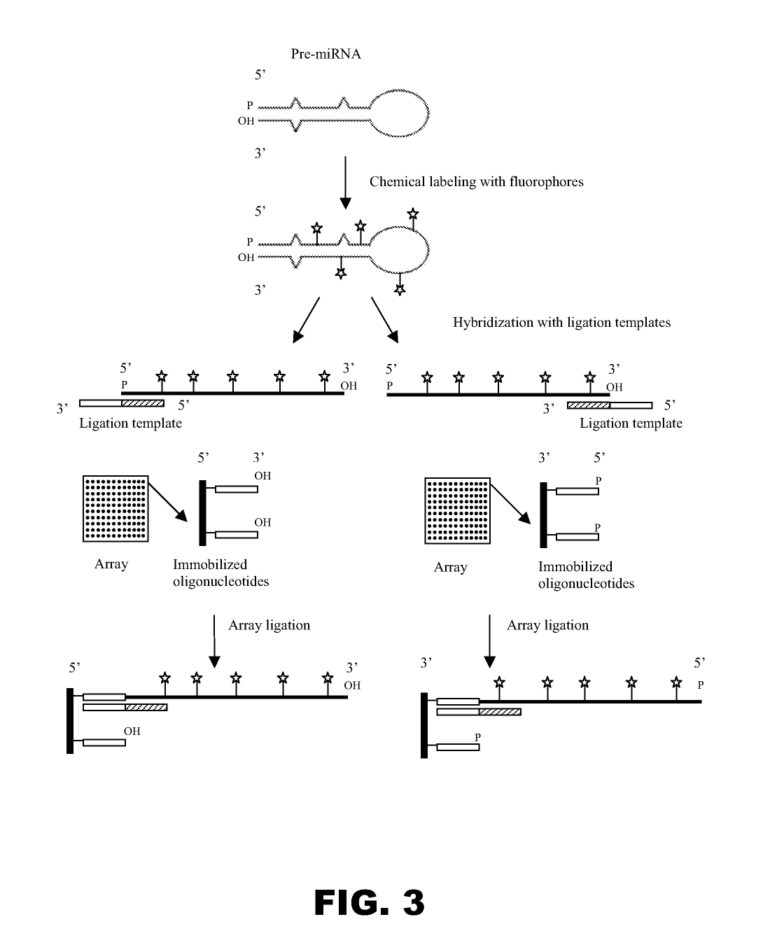

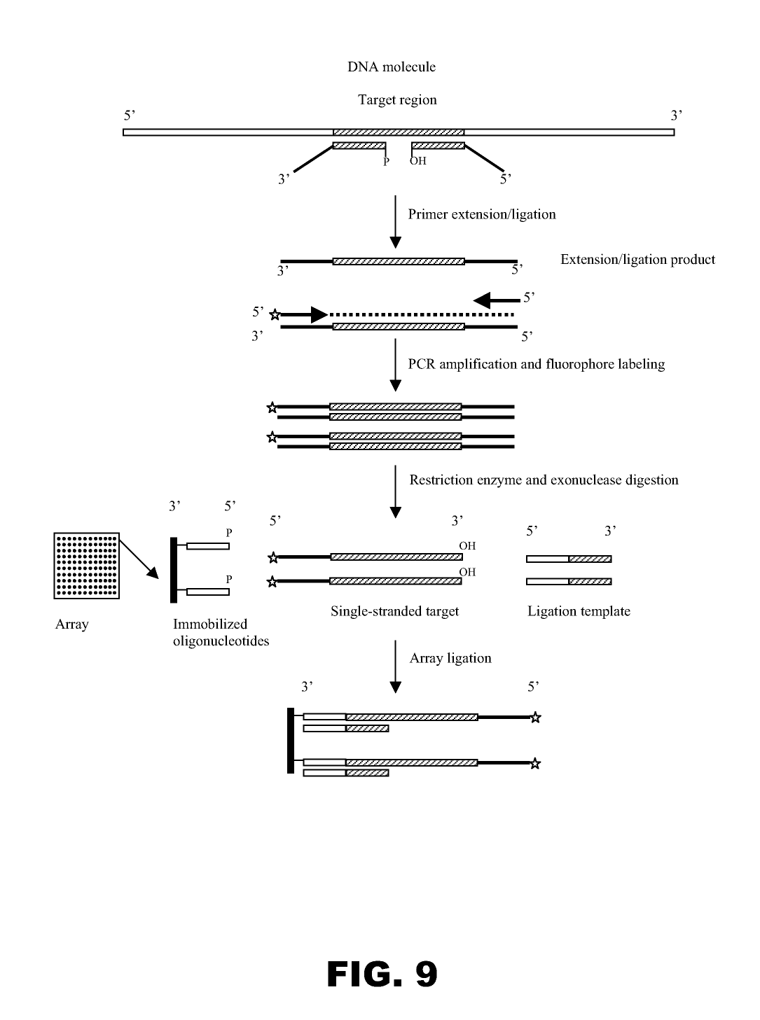

In some embodiments, at least one signaling molecule may be covalently attached to a target nucleic acid (e.g., see FIGS. 3-7). Those with skill in the art are familiar with chemical and enzymatic methods for coupling signaling molecules to nucleic acid molecules. For example, a signaling molecule may be attached to a target nucleic acid by alkylation using commercially available kits (e.g., from Mirus Bio Corporation, Madison, Wis.). The signaling molecule that is attached may be fluorophore (e.g., Cy3 or Cy5) or biotin. The method comprises contacting the target nucleic acid with reactive molecules, each of which comprises a signaling molecule, a positive charged linker, and an alkylating moiety, and covalently attaching the reactive molecules at N⁷ of guanine, N³ of adenine, or N³ of cytosine. On average, a signaling molecule may be attached every 20-60 bases. Alternatively, a signaling molecule may be attached to a target nucleic acid by ligating a 3′,5′-cytidine bisphosphate, which has a Cy dye attached to the 3′ phosphate, with the 3′ hydroxyl group of a target nucleic acid in a template independent ligation reaction (single strand ligation) catalyzed by T4 RNA ligase. Typically, the target nucleic acid is first dephosphorylated with a phosphatase, such as calf intestine alkaline phosphatase, to remove the 5′ terminal phosphate group prior to the single strand ligation reaction and prevent ligation between the target nucleic acids. The labeled target nucleic acid may be re-phosphorylated with a polynucleotide kinase, such as T4 polynucleotide kinase, before being analyzed by the method of the invention. In another embodiment, a target nucleic acid may be labeled by attaching a poly(A) tail to the target nucleic acid with a poly(A) polymerase, such as E. coli Poly(A) polymerase, and subsequently attaching signaling molecules (e.g., fluorescent dyes) to the poly(A) tail either by chemical or enzymatic means. In yet another embodiment, a signaling molecule may be attached to at least one of the primers used during PCR amplification of the target nucleic acid, such that the amplified target nucleic acid comprises at least one signaling molecule at one of its ends (e.g., see FIG. 9).

In still other embodiments, the signaling means may be attached to the target nucleic acid by the ligation of a “detection tag” (see FIGS. 1, 2, and 8). Mature small RNA molecules are generally labeled by the ligation of detection tags (see Examples 1-3). A detection tag comprises an oligonucleotide portion for ligation to a target nucleic acid and at least one signaling molecule. The signaling molecule may be a fluorescent dye, biotin, digoxigenin, or a sequence of nucleotides that is a target for branched DNA detection means. The oligonucleotide portion of a detection tag is complementary to a region of a ligation template, such that the ligation template facilitates the ligation of the detection tag to a target nucleic acid. The length of the oligonucleotide portion of a detection tag may be from about 6 to about 20 nucleotides in length. The signaling molecule may be attached to the 3′ end or the 5′ end of a detection tag (see FIGS. 1 and 2). If the 5′ end of a detection tag is free, then it will generally comprise a 5′ terminal phosphate group, such that it may be ligated to a target nucleic acid. Accordingly, a detection tag may be ligated to the 5′ end or the 3′ end of a target nucleic acid.

(iii) Contacting the Reactants

The order in which the different reactants are contacted can and will vary depending upon the application. In some embodiments, the target nucleic acids and the ligation templates may be contacted with the array of immobilized oligonucleotides concurrently. In other embodiments, the target nucleic acids may be contacted with the ligation templates prior to contact with the array of immobilized oligonucleotides. Contact between the target nucleic acids and the ligation templates may be at a constant temperature for a specific period of time. Alternatively, contact between the target nucleic acids and the ligation templates may be performed at a series of different temperatures for specific periods of time. For example, contact may comprise a temperature gradient such as 90° C. for 2 minutes, 60° C. for 10 minutes, 55° C. for 30 minutes, 50° C. for 30 minutes, and 45° C. for 10 minutes. In still other embodiments, the ligation templates may be contacted with the array of immobilized oligonucleotides, after which the array is contacted with the target nucleic acids. In other embodiments, more than one population of target nucleic acids may be contacted with an array of immobilized oligonucleotides. For example, a plurality of first target nucleic acids may be contacted with a plurality of first ligation templates to form a plurality of first hybridized products. Likewise, a plurality of second target nucleic acids may be contacted with a plurality of second ligation templates to form a plurality of second hybridized products. The pluralities of first and second hybridized products may be contacted with the array of immobilized oligonucleotides simultaneously or sequentially. One skilled in the art will appreciate that other iterations may be possible, especially when more than one population of nucleic acids is being analyzed.

After contact of the reactants, each ligation template may hybridize with its complementary specific immobilized oligonucleotide and its complementary target nucleic acid, whereby a specific target nucleic acid is held in close proximity to a specific immobilized oligonucleotide on the array.
(b) Ligating the Plurality of Target Nucleic Acids to the Immobilized Oligonucleotides on the Array

(i) Reaction Conditions

The method further comprises ligating the target nucleic acids to the immobilized oligonucleotides on the array. The ligation occurs in the presence of the ligation template, and the ligation is catalyzed by a template-dependent ligase. In general, ligation between two single-stranded nucleic acid molecules is more efficient in the presence of a complementary template strand. Ligation between the plurality of target nucleic acids and the plurality of immobilized oligonucleotide leads to the formation of a plurality of ligation products. Each ligation product comprises a target nucleic acid comprising a signaling means and an immobilized oligonucleotide that has a unique array position.

The polarity of the ligation reaction can and will vary, depending upon the orientation of the immobilized oligonucleotides on the array (see FIGS. 1-9). In some embodiments, the immobilized oligonucleotides may have free 3′ hydroxyl groups such that the 5′ end of the target nucleic acid may be ligated to the immobilized oligonucleotide. In other embodiments, the immobilized oligonucleotides may have free 5′ phosphate groups such that the 3′ end of the target nucleic acid may be ligated to the immobilized oligonucleotide.

A template-dependent ligase may form a phosphodiester bond between adjacent 3′ hydroxyl and 5′ phosphate groups in two DNA molecules, two RNA molecules, or a DNA molecule and an RNA molecule. The cofactor of the template-dependent ligase may be ATP or NAD. Non-limiting examples of suitable template-dependent ligases include mesophilic ligases such as T4 DNA ligase, T4 RNA ligase 2, vaccinia DNA ligase, E. coli DNA ligase, and a mammalian DNA ligase. Suitable template-dependent ligases also include thermophilic ligases such as Taq DNA ligase (from Thermus aquaticus), Tth DNA ligase (from Thermus thermophilus), Tfi DNA ligase (from Thermus filiformis), Pfu DNA ligase (from Pyrococcus furiosus), 9° N DNA ligase (from Thermococcus sp. strain 9° N), and Ampligase DNA ligase (available from Epicentre Biotechnologies, Madison, Wis.). In a preferred embodiment, the template-dependent ligase may be T4 DNA ligase. In another preferred embodiment, the template-dependent ligase may be 9° N DNA ligase. In another embodiment, the template-dependent ligase may comprise a combination of a mesophilic ligase and a thermophilic ligase.

Generally, the conditions of the ligation reaction will be adjusted such that the ligase functions near its optimal activity level. The pH utilized during the ligation reaction may range from about 6.5 to about 9.0, and more preferably from about 7.5 to about 8.5. A buffering agent may be utilized to adjust and maintain the pH at the desired level. Representative examples of suitable buffering agents include a Tris buffer, such as Tris-HCl, MOPS, HEPES, TAPS, Bicine, Tricine, TES, PIPES, and MES. In a preferred embodiment, the buffering agent may be Tris-HCl.

The ligation reaction mixture will generally comprise the appropriate cofactor (ATP or NAD). The concentration of the cofactor may vary, but generally will be within the optimal range.

The ligation reaction mixture may further comprise a divalent cation. Suitable divalent cations include calcium, magnesium, or manganese. In a preferred embodiment, the divalent salt may be magnesium chloride, manganese chloride, or a combination thereof. The concentration of the divalent salt may range from about 0.1 mM to about 15 mM, and preferably from about 1 mM to about 10 mM.

A monovalent cation may also be included in the ligation reaction mixture. Suitable monovalent cations include potassium, sodium, or lithium. In a preferred embodiment, the monovalent salt may be potassium chloride. The concentration of the monovalent salt may range from about 0.5 mM to about 100 mM, and preferably from about 1 mM to about 50 mM.

In one embodiment, the reaction mixture may further comprise a reducing agent. Non-limiting examples of suitable reducing agents include dithiothreitol and β-mercaptoethanol.

In another embodiment, the ligation reaction mixture may further comprise a ligation reaction enhancing polymer, such as PEG 4000.

In still another embodiment, the ligation reaction may optionally comprise an enzyme stabilizing molecule, such as bovine serum albumin (BSA).

In yet another embodiment, the ligation reaction mixture may optionally comprise a detergent, such as the nonionic surfactant Triton X-100.

The temperature of the ligation reaction will generally be adjusted such that the ligase functions near its optimal level. The temperature of the ligation reaction may range from about 14° C. to about 75° C. In one embodiment, the temperature of the reaction may range from about 30° C. to about 35° C. In another embodiment, the temperature of the reaction may range from about 35° C. to about 40° C. In still another embodiment, the temperature of the reaction may range from about 40° C. to about 45° C. In yet another embodiment, the temperature of the reaction may range from about 45° C. to about 50° C. In an alternate embodiment, the temperature of the reaction may range from about 50° C. to about 55° C. In another alternate embodiment, the temperature of the reaction may range from about 55° C. to about 65° C. Those of skill in the art will appreciate that ligation at a higher temperature will increase the hybridization stringency between the ligation template and the target sequences. The duration of the reaction will generally be long enough to allow completion of the reaction at a given temperature. In one embodiment, the duration of the reaction may range from about 4 hours to about 36 hours. In other embodiments, the duration of the reaction may be about 12 hours, about 14 hours, about 16 hours, about 18 hours, about 20 hours, about 22 hours, or about 24 hours.

(ii) Wash Conditions

The ligation reaction generally gives rise to a plurality of ligation products comprising target nucleic acids linked to immobilized oligonucleotides. Thus, the ligation products are covalently attached to the solid support. Upon completion of the ligation reaction, the array comprising the immobilized ligation products may be subjected to stringent wash conditions to remove all molecules that are not covalently attached to the solid support. Thus, non-target nucleic acid molecules and ligation templates will generally be removed, as well as any target nucleic acid molecule that was not successfully ligated. The covalently attached ligation products, however, will be retained. In an alternate embodiment, the array may be subjected to less stringent wash conditions to remove non-target nucleic acids and any target nucleic acid molecule that was not successfully ligated without removing the ligation templates, each of which facilitated the ligation between a target nucleic acids and an immobilized oligonucleotide and, thus, formed a stable duplex structure with the ligated product.

The stringent wash conditions may comprise elevated temperatures, such that double-stranded nucleic acid molecules are denatured. The elevated temperature may be about 50° C., about 60° C., about 70° C., about 80° C., about 90° C., or about 100° C. Additionally, the stringent wash conditions may comprise a solution of low ionic strength, such as provided by about 10 mM to about 100 mM NaCl, and/or an anionic detergent, such as SDS. Very stringent wash conditions may comprise a solution of extremely low ionic strength, such as deionized water. Furthermore, the stringent wash conditions may further comprise a chelating agent, such as EDTA, or denaturing agent, such as NaOH, urea, or formamide. The less stringent wash conditions may comprise lower temperatures and solutions of higher ionic strength.

In some embodiments, the array comprising the immobilized ligation products may be treated with a single-base mismatch cleavage enzyme prior to exposure to the wash conditions. Non-limiting examples of suitable single-base mismatch cleavage enzymes include RNase I, RNase A, and RNase T1. Without being bound by a particular theory, this treatment step may enhance the discrimination between nearly identical target nucleic acids or assist in single nucleotide mutation mapping.
(c) Quantifying the Signal

The method further comprises quantifying the signals associated with the ligation products, which are covalently attached to the array. Each ligation product comprises a signaling means. The signal generated by the signaling means may be detected by scanning the array and measuring fluorescence emission, fluorescence polarization, luminescence, chemiluminescence, phosphorescence, colorimetry, radioactivity, magnetism, electrochemistry, and the like. The scanning may be carried out by a microarrray scanner, a laser scanner, a multiphoton scanner, a flow cytometer, a charge-coupled device, a fluorimager, an electrochemiluminescent imager, a phosphor imager, a confocal microscope, a scanning electron microscope, an infrared microscope, an atomic force microscope, or an electrical conductance imager. Appropriate computer analysis programs and statistical programs may be used to correlate the measured signals with the presence, relative abundance, or absence of the target nucleic acid in a test sample.

Each ligation product (and consequently, each target nucleic acid) may be traced and identified not only by its signaling means, but also by its array position. For example, a target nucleic acid from test sample 1 may be labeled with Cy3 and the equivalent target nucleic acid from test sample 2 may be labeled with Cy5. The two target nucleic acids may be ligated to the same array position or to different array positions. Alternatively, a target nucleic acid from test sample 1 may be labeled with Cy3 and the equivalent target nucleic acid from test sample 2 may also be labeled with Cy3, and the two target nucleic acids are ligated to different array positions.
(d) Using the Method to Analyze Mature Small RNA Molecules

In one embodiment, the method of the invention may be used to analyze at least one population of mature small RNA molecules, as demonstrated in Examples 1 and 2. The analysis may comprise profiling the global expression patterns of populations of mature small RNAs, examining the expression levels of a specific mature small RNA, and so forth. The expression may be analyzed in equivalent test samples exposed to different conditions, equivalent test samples at different stages of the life cycle, or in different test samples, e.g., control cells vs. cancer cells. This method may also be used to discriminate between a mature small RNA and its precursor RNA. Mature small RNAs and their precursors may be distinguished on the basis of size, as detailed above.

In general, the plurality of target nucleic acids that is contacted with the array will be the plurality of mature small RNAs of interest. As detailed above in section (II)(a)(i), non-limiting examples of target mature small RNAs include mature miRNAs, mature siRNAs, mature rasiRNAs, mature tasiRNAs, mature piRNAs, and mature 21U-RNAs. In a preferred embodiment, the population of mature small RNAs may be a population of mature microRNAs. The sizes of the target mature small RNAs may vary, depending upon the class of mature small RNA molecules. Typically, the target mature small RNAs may range from about 16 nucleotides to about 40 nucleotides in length. Non-limiting examples of sources of target mature small RNAs that may be analyzed by the method of the invention include a total RNA preparation, a small RNA preparation, a microRNA preparation, a cell lysate, or a biological fluid.

In some embodiments, the signaling means of each target mature small RNA comprises a detection tag that is ligated to the target mature small RNA. Each detection tag comprises at least one signaling molecule selected from the group consisting of a fluorescent dye, biotin, digoxigenin, and a sequence of nucleotides that is a target for branched DNA detection. In a preferred embodiment, the signaling molecule is a fluorescent dye. Ligation of a detection tag to a target mature small RNA is catalyzed by a template-dependent ligase in the presence of a ligation template. Thus, each ligation template utilized in the analysis of mature small RNA molecules may also comprise a region with complementarity to the oligonucleotide portion of a detection tag (see FIGS. 1 and 2). A detection tag may be ligated to a target mature small RNA prior to its contact with and ligation to an immobilized oligonucleotide on an array. Alternatively, a detection tag may be ligated to the target mature small RNA simultaneously with the ligation of the target mature small RNA to an immobilized oligonucleotide on an array.

The method comprises contacting an array of immobilized oligonucleotides with a plurality of target small RNAs and a plurality of ligation templates. Each ligation template comprises a region that is complementary to the artificial sequence of an immobilized oligonucleotide, a region that is complementary to a target mature small RNA, and a region that is complementary to a portion of a detection tag. Hybridization between a particular ligation template and its complementary target mature small RNA, its complementary detection tag, and its complementary immobilized oligonucleotide directs a particular detection tag to the target mature small RNA and that same target mature small RNA to a particular immobilized oligonucleotide on an array (see FIGS. 1 and 2). The ligation template then facilitates these two ligation reactions, i.e., ligation between the target mature small RNA and the immobilized oligonucleotide and ligation between the target mature small RNA and the detection tag. The ligations are catalyzed by a template-dependent ligase. These ligation reactions give rise to a plurality of ligation products, wherein each ligation product comprises an immobilized oligonucleotide covalently linked to a target mature small RNA that is covalently linked to a detection tag. The method may also comprise subjecting the array comprising the immobilized ligation products to wash conditions to remove non-covalently attached molecule, such that the signals associated with each immobilized ligation product may be detected and quantified.

In other embodiments, the signaling means of each target mature small RNA may comprise at least one signaling molecule directly attached to the target mature small RNA. The signaling molecule may be a fluorescent dye, biotin, or digoxigenin, and the signaling molecule may be attached by chemical alkylation, single-strand nucleic acid ligation, or poly(A) extension, as described above in section (II)(a)(ii). Preferably, the signaling molecule is a fluorescent dye. In embodiments in which the target mature small RNA is directly labeled, each ligation template comprises two regions: a first with complementarity to the unique artificial sequence of an immobilized oligonucleotide and a second with complementarity to the target mature small RNA. Other features of the method are as described above.

The polarity of the ligation reactions may vary, depending upon the orientation of the immobilized oligonucleotides and the orientation of the ligation templates. In some embodiments, the 5′ end of a target mature small RNA may be ligated to an immobilized oligonucleotide (see Example 1 and FIG. 1). In other embodiments, the 3′ end of a target mature small RNA may be ligated to an immobilized oligonucleotide (see Example 2 and FIG. 2).

Sets of ligation templates may be engineered to detect all of the known mature small RNAs of a particular class of mature small RNAs in a particular organism. A set of ligation templates may have complementarity to one detection tag, or a set may comprise subsets of ligation templates, with each subset having complementarity to a different detection tag. Alternatively, multiple sets of ligation templates may be prepared for one population of mature small RNAs, with each set having complementarity to a different detection tag. Furthermore, a set of ligation templates may be easily expanded to include new ligation templates if new members of a certain class of mature small RNAs are discovered.
(e) Using the Method to Analyze Precursor Small RNA Molecules

In another embodiment, the method of the invention may be used to analyze at least one population of precursor small RNAs. The analysis may comprise profiling the global expression patterns of populations of precursor small RNAs or examining the expression levels of a specific precursor small RNA. For example, the expression patterns of precursor small RNAs may be correlated with those of their mature small RNAs for analysis of the post-transcriptional regulation patterns of mature small RNAs. In general, the plurality of target nucleic acids that is contacted with an array of immobilized oligonucleotides will be the plurality of precursor small RNAs. In a preferred embodiment, the plurality of precursor small RNAs may be a plurality of precursor microRNAs (pre-miRNAs).

The sizes of the target precursor small RNAs may vary, depending upon the class of precursor small RNA molecules. Typically, the target precursor small RNAs may range from about 50 nucleotides to about 160 nucleotides in length. Non-limiting examples of sources of target precursor small RNAs that may be analyzed by the method of the invention include a total RNA preparation, a small RNA preparation, a precursor small RNA preparation, a cell lysate, or a biological fluid

The signaling means of a plurality of precursor small RNAs may be at least one directly attached signaling molecule, as detailed above for mature small RNAs. In a preferred embodiment, the signaling molecule is a fluorescent dye. The method comprises denaturing and contacting a plurality of target precursor small RNAs with a plurality of ligation templates, such that a plurality of adaptor-like hybridization products are formed between each target precursor small RNA and its complementary ligation template. The method further comprises contacting the plurality of hybridization products with a plurality of immobilized oligonucleotides on an array and ligating the plurality of target precursor small RNAs to the plurality of immobilized oligonucleotides. Each ligation template comprises a region that is complementary to a target precursor small RNA and a region that is complementary to the unique artificial sequence of an immobilized oligonucleotide. The method may also comprise subjecting the array comprising the immobilized ligation products to stringent wash conditions to remove non-covalently attached molecules, such that the signals associated with each immobilized ligation product may be detected and quantified.

The polarity of the ligation reactions may vary. In some embodiments, the 3′ end of a target precursor small RNA may be ligated to an immobilized oligonucleotide. In other embodiments, the 5′ end of a target precursor small RNA may be ligated to an immobilized oligonucleotide (see FIG. 3). In still other embodiments in which a plurality of precursor small RNAs and a plurality of their mature small RNAs are analyzed simultaneously on an array, the orientation of a ligation template for a precursor small RNA may vary depending on the location of the mature small RNA sequence within the precursor small RNA. A ligation template for a precursor small RNA may comprise a region that is complementary to the 5′ end region of the precursor small RNA if the mature small RNA sequence is located in the 3′ region of the precursor small RNA, and a ligation template for a precursor small RNA may comprise a region that is complementary to the 3′ end region of the precursor small RNA if the mature small RNA sequence is located in the 5′ region of the precursor small RNA. Accordingly, an array may comprise oligonucleotides immobilized in their 3′ ends in some array positions and oligonucleotides immobilized in their 5′ ends in other array positions.

Sets of ligation templates may be engineered to detect all of the known precursor small RNAs of a particular class of precursor small RNAs in a particular organism. Furthermore, a set of ligation templates may be easily expanded to include new ligation templates if new members of a certain class of precursor small RNAs are discovered.
(f) Using the Method to Analyze Messenger RNA Molecules

In yet another embodiment, the method of the invention may be used to analyze at least one population of messenger RNA molecules. The analysis may comprise profiling the global expression patterns of populations of messenger RNAs or examining the expression levels of a specific messenger RNA. The analysis may also comprise profiling the expression patterns of a selected group of messenger RNAs that are involved in a certain pathway of interest or in a disease state. Non-limiting examples of messenger RNAs include poly(A)⁺ and poly(A)⁻ messenger RNAs from eukaryotes and messenger RNAs from prokaryotes that are typically without a poly(A) tail.

Typically, the plurality of target nucleic acids that is contacted with an array of immobilized oligonucleotides is the population of messenger RNA molecules or fragments thereof. Alternatively, the population of messenger RNAs may be first converted to a population of cDNA molecules and the population of the cDNA molecules may be then converted to a population of cRNA molecules before being analyzed by the method of the invention. The messenger RNAs that are contacted with an array may range from about 100 nucleotides to about 10,000 nucleotides in length, and the messenger RNA fragments that are contacted with an array may range from about 100 nucleotides to about 5,000 nucleotides in length. Non-limiting examples of sources of messenger RNAs that may be analyzed by the method of the invention include a total RNA preparation, a messenger RNA preparation, a poly(A)⁺ messenger RNA preparation, a cell lysate, or a biological fluid

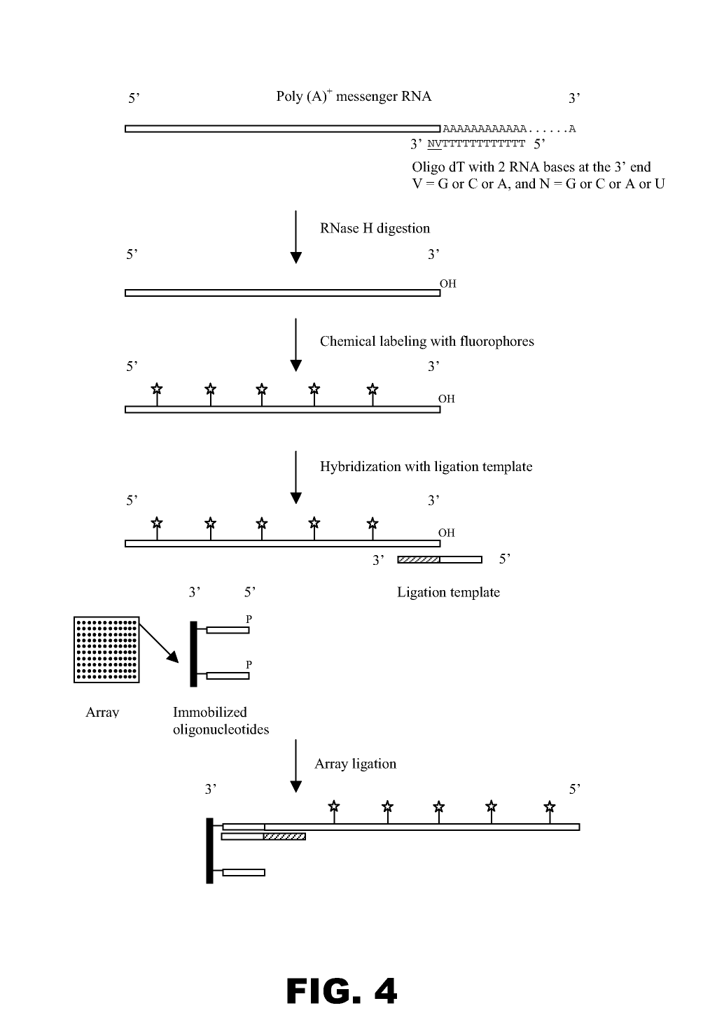

In one embodiment, the method comprises removing the poly(A) tails from target poly(A)⁺ messenger RNAs and generating fragments with gene-specific 3′ ends with 3′ hydroxyl groups (see FIG. 4). The method comprises contacting each poly(A)⁺ messenger RNA with an anchor oligo dT primer to form an RNA/DNA heteroduplex and digesting the RNA/DNA heteroduplex with an RNase H. The anchor oligo dT primer may comprise (5′ to 3′) a string of deoxythymidylic acid (dT) residues followed by two additional ribonucleotides represented by VN, wherein V is either G, C, or A and N is either G, C, A, or U. The VN ribonucleotide anchor allows the primer to hybridize only at the 5′ end of the poly(A) tail of a target messenger RNA. Accordingly, the anchor RNA/oligo dT primer is a pool of 12 oligo RNA/dT primers, each with a different pair of ribonucleotides at the 3′ end. The number of dT residues in each anchor primer may range from about 12 to about 20. The anchor oligo dT primer may optionally comprise a biotin whereby the primer may be used to purify or enrich poly(A)⁺ messenger RNAs before RNase H digestion. RNase H specifically hydrolyzes the phosphodiester bonds of RNA that is hybridized to DNA. The RNase H may be a native or recombinant enzyme isolated from a mesophilic organism, such as E. coli RNase H that is available from several commercial suppliers. Alternatively, the RNase H may be a native or recombinant enzyme isolated from a thermophilic organism, e.g., Hybridase™ Thermostable RNase H (available from Epicentre Biotechnologies). As a result of the RNase H digestion, each target messenger RNA fragment generally comprises a gene-specific 3′ end with a 3′ hydroxyl group. In other embodiments, the anchor oligo dT primer may comprise (5′ to 3′) a string of dT residues followed by two, three, or four additional deoxyribonucleotides represented by VN, VNN, or VNNN, wherein V is dG, dC, or dA and N is dG, dC, dA, or dT. Accordingly, the anchor DNA/oligo dT primer may comprise a pool of 12 oligo dT primers, a pool of 48 oligo dT primers, or a pool of 192 oligo dT primers. In these configurations, two, three, or four additional ribonucleotides may be removed by RNase H digestion from the 3′ end of each target messenger RNA, in addition to the poly(A) tail. As a result of the RNase H digestion, each target messenger RNA fragment generally comprises a gene-specific 3′ end with a 3′ hydroxyl group, with the 3′ end corresponding to a position that was two, three, or four nucleotides upstream from the 5′ end of the poly(A) tail. Those of skill in the art will appreciate that other iterations are possible.

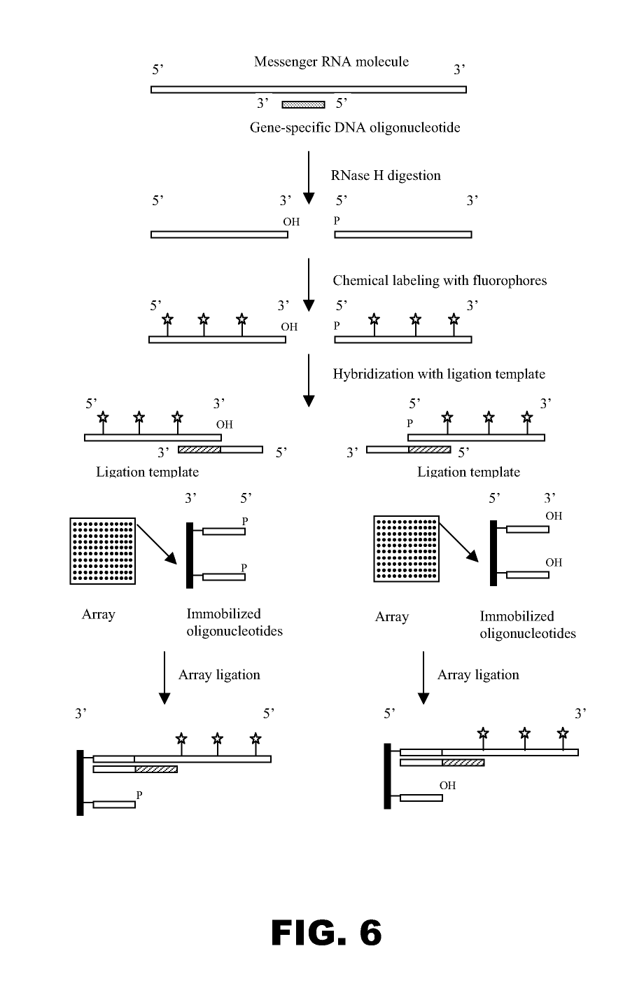

In an alternative embodiment, the method comprises fragmenting each target messenger RNA to generate a first target messenger RNA fragment with a nascent gene-specific 5′ end and a 5′ terminal phosphate group and a second target messenger RNA fragment with a nascent gene-specific 3′ end and 3′ hydroxyl group (see FIG. 6). This method may be used to distinguish between closely related target messenger RNAs or between the mutated form and the normal form of a messenger RNA that differ from each other by only one or a few nucleotides. This method may also be used to fragment very large messenger RNAs. Furthermore, this method may be used to generate target nucleic acids for messenger RNAs whose sequences are only partially known, e.g., the expressed sequence tags (ESTs). The method comprises contacting a target messenger RNA with a gene-specific DNA oligonucleotide to form an RNA/DNA heteroduplex and digesting the RNA/DNA heteroduplex with an RNase H, as detailed above. The resultant fragments may range from about 20 nucleotides to about 5,000 nucleotides in length.

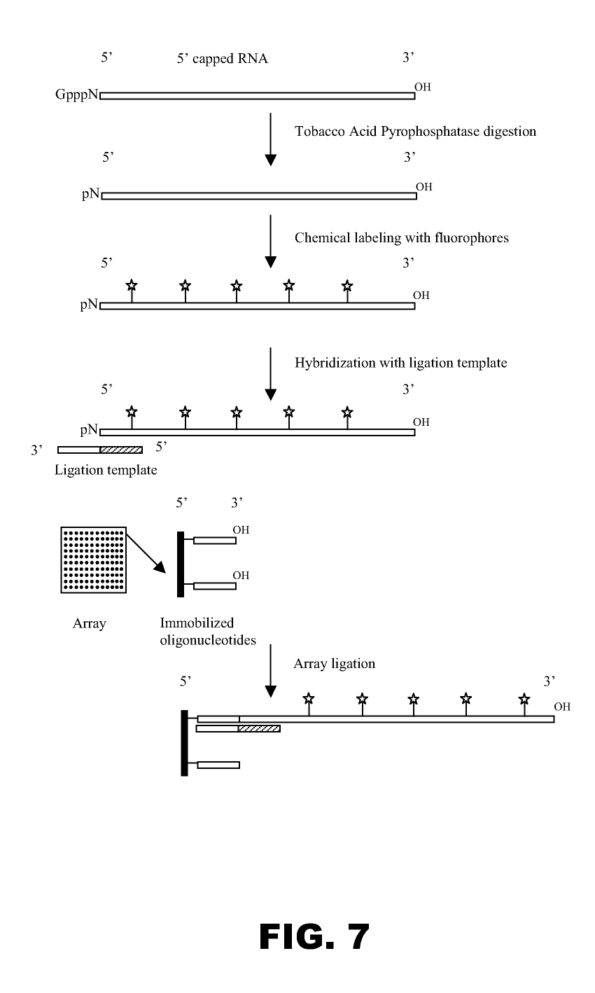

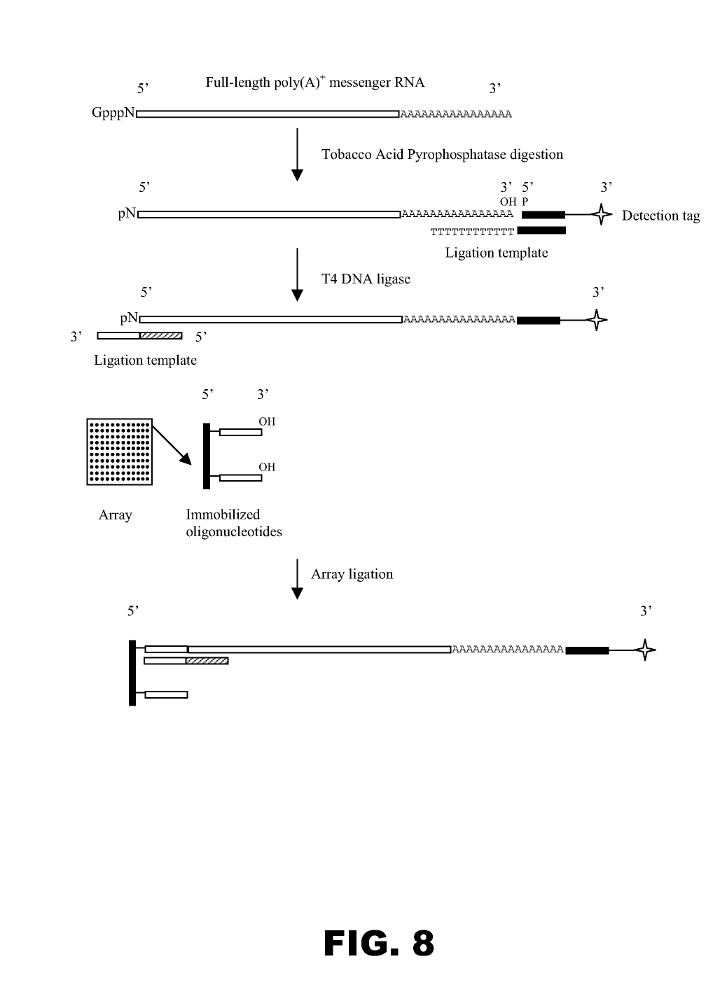

In further embodiments, the method comprises removing the 5′ cap structure of each target eukaryotic messenger RNA molecule or removing the pyrophosphate group in the triphosphate group of each target prokaryotic messenger RNA molecule to generate a 5′ terminal phosphate group at the first nucleotide of the molecule (see FIG. 7). This method may be used to map the transcription initiation sites of target messenger RNAs. Furthermore, this method may be used to analyze the expression patterns of full-length poly(A)⁺ messenger RNAs (FIG. 8). The method comprises digesting target messenger RNAs with a tobacco acid pyrophosphatase (TAP) enzyme to hydrolyze the phosphoric acid anhydride bonds in the 5′ cap structure of eukaryotic messenger RNAs or in the triphosphate group at the 5′ end of prokaryotic messenger RNAs. The resultant messenger RNAs may range from about 500 nucleotides to about 10,000 nucleotides in length.

The signaling means of each target messenger RNA molecule or fragment thereof comprises at least one signaling molecule that was enzymatically or chemically attached to the RNA molecule or fragment thereof. The signaling molecule may be a fluorescent dye, biotin, or digoxigenin. In a preferred embodiment, the signaling molecule is a fluorescent dye, such as Cy3 or Cy5.

The method further comprises contacting a plurality of target messenger RNAs or fragments thereof with a plurality of ligation templates, such that a plurality of adaptor-like hybridization products is formed between each target messenger RNA or fragment thereof and its complementary ligation template (FIGS. 4-8). The plurality of hybridization products is then contacted with a plurality of immobilized oligonucleotides on an array, whereby a plurality of ligation products is formed by a template-dependent ligase. Alternatively, the method may comprise contacting a plurality of immobilized oligonucleotides with a plurality of target messenger RNA or fragments thereof and a plurality of ligation templates without first forming the plurality of adaptor-like hybridization products.

The polarity of ligation templates may vary depending on how the target messenger RNAs are prepared. In some embodiments, the target messenger RNAs may be prepared by removing the poly(A) tails or the target messenger RNAs are naturally occurring poly(A)⁻ messenger RNAs, and each ligation template has (5′ to 3′) a first region that is complementary to the unique artificial sequence of an immobilized oligonucleotide and a second region that is complementary to the 3′ end region of a particular target messenger RNA. In other embodiments, the target messenger RNAs may be prepared by gene-specific fragmentation, and each ligation template has (5′ to 3′) a first region that is complementary to the unique artificial sequence of an immobilized oligonucleotide and a second region that is complementary to the 3′ end region of a particular target messenger RNA fragment, or each ligation fragment has (5′ to 3′) a first region that is complementary to the 5′ end region of a particular target messenger RNA fragment and a second region that is complementary to the unique artificial sequence of an immobilized oligonucleotide. In still other embodiments, the target messenger RNAs may be prepared by removing the 5′ cap structure or the 5′ pyrophosphate group, and each ligation template has (5′ to 3′) a first region that is complementary to the 5′ end region of a particular target messenger RNA and a second region that is complementary to the unique artificial sequence of an immobilized oligonucleotide.

Sets of ligation templates may be engineered to detect all of the known messenger RNAs of a particular organism. Subsets of ligation templates may be engineered to detect different groups of messenger RNAs that are involved in certain pathways or in certain disease states. Furthermore, a set of ligation templates may be easily expanded to include new ligation templates if new messenger RNAs are discovered.
(g) Using the Method to Analyze cDNA or Genomic DNA Molecules

In another alternate embodiment, the method of the invention may be used to analyze cDNA molecules or genomic DNA molecules. Accordingly, the plurality of target nucleic acids that is contacted with an array corresponds to regions of interest in cDNA molecules or genomic DNA molecules. The region of interest in a cDNA molecule may be, but is not limited to, a splice site, an alternate splice site, an alternative transcriptional start site, an alternative polyadenylation site, a region in a 5′ untranslated region (UTR), a region in a 3′ UTR, an edited region, or a polymorphic region. The region of interest in a genomic DNA molecule includes, but is not limited to, a single nucleotide polymorphism, a single point mutation, a methylated site, a transcription factor binding site, a small insertion, a small deletion, a small translocation, a single tandem repeat, and a small variable number of tandem repeats.

The method comprises contacting an array of immobilized oligonucleotides with a plurality of target DNA molecules, each of which corresponds to a region of interest in a cDNA or a genomic DNA molecule, and a plurality of ligation templates. The target DNA molecules that are contacted with an array may range from about 50 nucleotides to about 500 nucleotides in length. Each target DNA molecule comprises at least one signaling molecule attached to the DNA molecule. The signaling molecule may be a fluorescent dye, biotin, digoxigenin, or a sequence of nucleotides that is a target for branched DNA detection means. The signaling molecule may be attached to an amplification primer and incorporated into the target DNA molecule during a PCR amplification step (see FIG. 9). Each of the ligation templates used in this embodiment comprises a region that is complementary to the unique artificial sequence of an immobilized oligonucleotide and a region that is complementary to one region of a target DNA molecule. Hybridization between a ligation template and its complementary target DNA molecule and its complementary immobilized oligonucleotide guides the target DNA molecule to a particular array position. Furthermore, each ligation template facilitates the ligation between the target DNA molecule and the immobilized oligonucleotide, thereby forming a plurality of immobilized ligation products, which may be detected as described above

In one embodiment, the method may be used to analyze regions of interest in cDNA or genomic DNA molecules. Preparation of a target DNA molecule that is derived from a cDNA or genomic DNA molecule may comprise hybridizing a pair of oligonucleotides to the region of interest in the cDNA or genomic DNA molecule (see FIG. 9). The pair of oligonucleotides hybridized to the region of interest may be separated by a gap or they may be adjacent such that only a nick (i.e., no phosphodiester bond) separates them. The gap between the two oligonucleotides can and will vary depending on the application. In general, the gap may range from about one nucleotide to about 20 nucleotides in length. The oligonucleotide downstream of the gap/nick may comprise a terminal phosphate at the 5′ end and a universal priming site at the 3′ end. The oligonucleotide upstream of the gap/nick may comprise a terminal hydroxyl group at the 3′ end and a universal priming site at the 5′ end. Non-limiting examples of suitable universal priming sites include T7 promoter sequence, T3 promoter sequence, SP6 promoter sequence, M13 forward sequence, M13 reverse sequence, or essentially any artificial sequence that is not present in the target nucleic acid. One of the oligonucleotides may further comprise a restriction endonuclease recognition site within the universal priming site. In a preferred embodiment, the upstream oligonucleotide comprises a restriction enzyme recognition site. Examples of preferred restriction endonucleases include AarI, BspQI, BspTNI, and SapI, which cleave duplex DNA downstream of the recognition site and leave a 5′ terminal phosphate at the cleavage site. Other types of restriction endonucleases may also be used.

After hybridization, the excess oligonucleotides may be removed by a method known to those of skill in the art. If the oligonucleotides are separated by a gap, then a primer extension assay may be used to fill in the gap. The primer extension reaction may be catalyzed by a DNA polymerase, such as Klenow Fragment, which lacks 5′ to 3′ exonuclease activity. If a nick separates the oligonucleotides, then the primer extension step may be omitted. The pair of oligonucleotides may be ligated together to form a ligation product. The ligation reaction is catalyzed by a template-dependent ligase, as detailed above in section (II)(b).

The ligation product may be PCR amplified using the appropriate pair of universal primers. The universal primer corresponding to the oligonucleotide that does not contain the restriction site may be labeled with a signaling molecule, such as a fluorescent dye, at the 5′ end. Thus, PCR amplification will generate a labeled target DNA molecule corresponding to the region of interest of the cDNA molecule or the genomic DNA molecule. After PCR amplification, the amplified target DNA molecule may be digested with the appropriate restriction enzyme and a 5′-phosphate-dependent exonuclease, such as Terminator™ Exonuclease (Epicentre Biotechnologies). These two digestions may be performed simultaneously or sequentially. The restriction endonuclease may cleave the end of the amplified target DNA molecule that contains its recognition restriction site in the primer region, leaving a 5′ terminal phosphate in the unlabeled strand of the amplified product. The 5′-phosphate-dependent exonuclease may then selectively degrade the unlabeled strand of the amplified product. The resultant labeled, single-stranded molecule is a target DNA molecule that may be contacted with an array of immobilized oligonucleotides in the presence of the appropriate ligation templates (as shown in FIG. 9).

In another embodiment, the method may be used to analyze the methylation status of specific CpG islands or specific CpG sites in genomic DNA. The target DNA molecules may be prepared by first treating the genomic DNA with bisulfite. This treatment converts unmethylated C residues to U residues (or T residues after the subsequent PCR amplification). After the C to T conversion, the complementarity between the two strands is lost and the overall sequence complexity is reduced. Therefore, it may be necessary to attach a unique identifier sequence to the region of interest to increase sequence complexity (see below). Then the region of interest may be hybridized with two pairs of oligonucleotides in two separate reactions. One pair of oligonucleotides is designed for methylated DNA and the other pair is designed for unmethylated DNA. A nick or a gap may separate each pair of hybridized oligonucleotides, as described above. The 3′ end of the upstream oligonucleotide and/or the 5′ end of the downstream oligonucleotide may align with a CpG site, and preferably, the 3′ end of the upstream oligonucleotide and/or the 5′ end of the downstream oligonucleotide may each align with a C in a CpG site. The oligonucleotides of each pair may comprise universal priming sites, a restriction endoncuclease recognition site, and optionally, a unique identifier sequence, as described above. Primer extension, ligation, PCR amplification, endonuclease digestion, and exonuclease digestion may be conducted as described above. Methylated and unmethylated targets may be differentially labeled during PCR, i.e., via the incorporation of different signaling molecules into the appropriate universal primer. The resultant labeled, single-stranded target DNA molecule may be contacted with and ligated to an array of immobilized oligonucleotides in the presence of the appropriate ligation templates, as described above.

In still another embodiment, the method may be used to analyze single nucleotide polymorphisms (SNPs) and/or single point mutations in genomic DNA. For each nucleotide to be queried, up to four pairs of complementary oligonucleotides may be designed (with each pair comprising universal priming sites and a restriction endonuclease recognition site, as described above). Each pair also comprises a different nucleotide at the position corresponding to the nucleotide of interest. The target genomic DNA comprising the nucleotide of interest may be hybridized with each of four pairs of oligonucleotides in separate reactions. Each pair of oligonucleotides may be separated on the target region by a nick or a gap. If a gap separates a pair of oligonucleotides, then the 3′ terminal nucleotide of the upstream oligonucleotide may align with the target nucleotide of interest. If a nick separates a pair of oligonucleotides, then the 3′ end of the upstream oligonucleotide or the 5′ end of the downstream oligonucleotide may align with the target nucleotide of interest. Primer extension, ligation, PCR amplification, endonuclease digestion, and exonuclease digestion may be conducted, as described above. Products from the four ligation reactions may be differentially labeled during PCR, such that each comprises a different signaling molecule. The amplified target DNA molecules may then be contacted with the array, as detailed above. Those of skill in the art will appreciate that other iterations of the method of the invention may be used to analyze other types of genomic polymorphisms.
(h) Using the Method to Analyze Mature Small RNAs, Precursor Small RNAs, Messenger RNAs, and Genomic Variations Simultaneously

The method of the invention may further be used for the analysis of the expression patterns of mature small RNAs, precursor small RNAs, messenger RNAs and fragments thereof, as well as the variations of cDNA and genomic DNA molecules simultaneously on an array of immobilized oligonucleotides. The array may comprise oligonucleotides that are immobilized by their 5′ ends in some positions and oligonucleotides that are immobilized by their 3′ ends in other positions. Each sub-population of target nucleic acids may be prepared and hybridized to their complementary ligation templates independently. The method further comprises pooling the sub-populations of the hybridized target nucleic acids/ligation templates and contacting an array of immobilized oligonucleotides with the pool of hybridized products and forming a plurality of ligation products with a template dependent ligase. Other features of the method are as described above.

### (III) Kit for Analyzing at Least One Population of Nucleic Acids

A further aspect of the present invention provides a kit for analyzing at least one population of nucleic acids. The kit comprises an array of immobilized oligonucleotides, each comprising a unique artificial sequence, which was described in section (I)(a), a plurality of ligation templates, which was described in section (I)(b), and a template-dependent ligase, which was described in section (II)(b). The kit may further comprise at least one detection tag or a signaling molecule, both of which were described in section (II)(a)(i).

### Definitions

To facilitate understanding of the invention, a number of terms are defined below.

As used herein, the terms “complementary” or “complementarity” refer to the association of double-stranded nucleic acids by standard base pairing through specific hydrogen bonds (i.e., 5′-A G T C-3′ pairs with the complimentary sequence 3′-T C A G-5′). Complementarity between two single-stranded molecules may be partial, if only some of the nucleic acid pairs are complimentary, or complete, if all the base pairs are complimentary.

The term “hydrocarbyl” as used herein describes organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “hybridization,” as used herein, refers to the process of hydrogen bonding, or base pairing, between the bases comprising two complementary single-stranded nucleic acid molecules to form a double-stranded hybrid. The “stringency” of hybridization is typically determined by the conditions of temperature and ionic strength. Nucleic acid hybrid stability is generally expressed as the melting temperature or Tₘ, which is the temperature at which the hybrid is 50% denatured under defined conditions. Equations have been derived to estimate the Tₘ of a given hybrid; the equations take into account the G+C content of the nucleic acid, the nature of the hybrid (e.g., DNA:DNA, DNA:RNA, etc.), the length of the nucleic acid probe, etc. (e.g., Sambrook et al. (1989) Molecular Cloning: A Laboratory Manual, Cold Spring Harbor Press, Cold Spring Harbor, N.Y., chapter 9). In many reactions that are based upon hybridization, e.g., polymerase reactions, amplification reactions, ligation reactions, etc., the temperature of the reaction typically determines the stringency of the hybridization.

The term “oligonucleotide,” as used herein, refers to a molecule comprising two or more nucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos. The nucleotides may be linked by phosphodiester, phosphothioate, phosphoramidite, or phosphorodiamidate bonds.

The term “substituted hydrocarbyl” used herein refers to hydrocarbyl moieties that are substituted with at least one atom, including moieties in which a carbon chain atom is substituted with a hetero atom such as nitrogen, oxygen, silicon, phosphorous, boron, sulfur, or a halogen atom. These substituents include halogen, heterocyclo, hydrocarbyloxy such as alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

The term “target nucleic acid,” as used herein, refers to a single-stranded nucleic acid that hybridizes with a ligation template and is ligated to an immobilized oligonucleotide on an array. A target nucleic acid may be all of or a part of a nucleic acid molecule, or it may be derived from a nucleic acid molecule (e.g., a cDNA copy).

As used herein, the term “unique artificial sequence” refers to a randomly generated nucleotide sequence with no intended complementarity to that of any known organism.

The term “universal array,” as used herein, refers to an array of oligonucleotides comprising artificial sequences, i.e., sequences that are not dependent on any complementarity to those of any organism for analysis of a population of target nucleic acids of any organism. Accordingly, a universal array may be adapted for use with any organism or any population of target nucleic acids.

### EXAMPLES

The following examples are included to demonstrate various embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes may be made in the specific embodiments that are disclosed and still obtain a like or similar result without departing from the spirit and scope of the invention, therefore all matter set forth in the above description and in the examples given below, shall be interpreted as illustrative and not in a limiting sense.

### Example 1. Ligation Array Analyses Using Immobilized Oligonucleotides with Free 3′ Hydroxyl Groups

The purpose of this experiment was to evaluate whether the 5′ terminal phosphate group of an RNA molecule may be ligated to the free 3′ hydroxyl group of an oligonucleotide immobilized on a solid support via the catalytic activity of a template-dependent ligase in the presence of a ligation template, as depicted in FIG. 1. The RNA molecules to be analyzed were human mature microRNAs, and their expression levels were analyzed in two different human cell lines.

(i) Array of Immobilized Oligonucleotides

All of the oligonucleotides used in this example were synthesized by conventional techniques. Each of the oligonucleotides to be immobilized on glass slides was either 10 or 20 nucleotides in length: each comprised a unique artificial sequence of 10 nucleotides, with 50% GC content, and some further comprised an extension of 10 adenosine residues (As) at the 5′ end. Each oligonucleotide was also modified with a C12-amine group at the 5′ end during synthesis. In addition, four of the oligonucleotides were modified with a ribonucleotide at the 3′ end. The sequences of the immobilized oligonucleotides are presented in Table 1.

Each oligonucleotide sample was first dissolved in nuclease-free water at 250 μM and then diluted to 50 μM in 100 mM sodium phosphate, pH 8.5. Each sample was printed onto CodeLink slides (Amersham Biosciences; Piscataway, N.J.) in five locations in each array with a GMS 417 Arrayer instrument (Genetic MicroSystems Inc.; Woburn, Mass.). Each slide was printed with four arrays of the oligonucleotides. Printed slides were placed in a humidity chamber containing a saturated NaCl solution for 20 hours. The saturated NaCl solution provided about 75% relative humidity, which was desirable for the covalent linkage reaction between the amine group at the 5′ end of each oligonucleotide and an N-hydroxysuccinimide (NHS) ester reactive group on the slide surface. Slides were then placed in a blocking solution (0.1 M Tris, 50 mM ethanolamine, pH 9.0), which had been pre-warmed to 50° C., for 30 minutes on a shaker to block residual N-hydroxysuccinimide (NHS) ester reactive groups, and washed in 4×SSC, 0.1 SDS, pre-warmed to 50° C., for 30 minutes on a shaker. Subsequently, the slides were thoroughly washed in deionized water and dried by centrifugation at 1,000 rpm for 3 minutes.

(ii) Ligation Templates and Detection Tags

Two sets of ligation templates were synthesized for analyzing 24 human mature microRNAs. Each of the 24 microRNAs was analyzed by a pair of templates that differed only in the region that was complementary to a detection tag. Each ligation template comprised (5′ to 3′) a first region (10 nucleotides in length) that was complementary to an oligonucleotide portion of a detection tag, a second region that was complementary to a human mature microRNA, and a third region (10 nucleotides in length) that was complementary to the unique artificial sequence of a particular immobilized oligonucleotide. The first set of ligation templates (Set A) comprised the first region that was complementary to a Cy3 detection tag and the second set of ligation templates (Set B) comprised the first region that was complementary to a Cy5 detection tag. The Cy3 detection tag comprised 5′-gacaactgactgatactcta-Cy3 (SEQ ID NO: 1), and the Cy5 detection tag comprised 5′-cgtgtgatgatgatactcta-Cy5 (SEQ ID NO: 2). Each detection tag comprised a 5′ terminal phosphate group. The ligation templates in each set were combined to form a pool. The target microRNAs, immobilized oligonucleotides, and their correspondent ligation templates are presented in Table 1.

TABLE 1

Target Mature MicroRNAs, Ligation Templates, and Immobilized
Oligonucleotides Used in Example 1.

| | | |
|---|---|---|
| Name | Sequence (5′ to 3′)* | SEQ ID NO: |

| | | |
|---|---|---|
| hsa-let-7a | UGAGGUAGUAGGUUGUAUAGUU | 3 |
| Lig. Temp. 1A | gtcagttgtcaactatacaacctactacctcactccctcttt | 4 |
| Lig. Temp. 1B | tcatcacacgaactatacaacctactacctcactccctcttt | 5 |
| Immob. Oligo 1 | aaagagggag | 6 |
| hsa-miR-16 | UAGCAGCACGUAAAUAUUGGCG | 7 |
| Lig. Temp. 2A | gtcagttgtccgccaatatttacgtgctgctacatcgccttt | 8 |
| Lig. Temp. 2B | tcatcacacgcgccaatatttacgtgctgctacatcgccttt | 9 |
| Immob. Oligo 2 | aaaggcgatg | 10 |
| hsa-miR-21 | UAGCUUAUCAGACUGAUGUUGA | 11 |
| Lig. Temp. 3A | gtcagttgtctcaacatcagtctgataagctaccctcgattt | 12 |
| Lig. Temp. 3B | tcatcacacgtcaacatcagtctgataagctaccctcgattt | 13 |
| Immob. Oligo 3 | aaatcgaggg | 14 |
| hsa-miR-23b | AUCACAUUGCCAGGGAUUACC | 15 |
| Lig. Temp. 4A | gtcagttgtcggtaatccctggcaatgtgatcggaccattt | 16 |
| Lig. Temp. 4B | tcatcacacgggtaatccctggcaatgtgatcggaccattt | 17 |
| Immob. Oligo 4 | aaatggtccg | 18 |
| hsa-miR-29a | UAGCACCAUCUGAAAUCGGUU | 19 |
| Lig. Temp. 5A | gtcagttgtcaaccgatttcagatggtgctagccctttgtt | 20 |
| Lig. Temp. 5B | tcatcacacgaaccgatttcagatggtgctagccctttgtt | 21 |
| Immob. Oligo 5 | aacaaagggc | 22 |
| hsa-miR-29b | UAGCACCAUUUGAAAUCAGUGUU | 23 |
| Lig. Temp. 6A | gtcagttgtcaacactgatttcaaatggtgctatcggtgtgtt | 24 |
| Lig. Temp. 6B | tcatcacacgaacactgatttcaaatggtgctatcggtgtgtt | 25 |
| Immob. Oligo 6 | aacacaccga | 26 |
| hsa-miR-30b | UGUAAACAUCCUACACUCAGCU | 27 |
| Lig. Temp. 7A | gtcagttgtcagctgagtgtaggatgtttacaagtgtcggtt | 28 |
| Lig. Temp. 7B | tcatcacacgagctgagtgtaggatgtttacaagtgtcggtt | 29 |
| Immob. Oligo 7 | aaccgacact | 30 |
| hsa-miR-31 | GGCAAGAUGCUGGCAUAGCUG | 31 |
| Lig. Temp. 8A | gtcagttgtccagctatgccagcatcttgcctaacgcggtt | 32 |
| Lig. Temp. 8B | tcatcacacgcagctatgccagcatcttgcctaacgcggtt | 33 |
| Immob. Oligo 8 | aaccgcgtta | 34 |
| hsa-miR-34a | UGGCAGUGUCUUAGCUGGUUGUU | 35 |
| Lig. Temp. 9A | gtcagttgtcaacaaccagctaagacactgccagagagaggtt | 36 |
| Lig. Temp. 9B | tcatcacacgaacaaccagctaagacactgccagagagaggtt | 37 |
| Immob. Oligo 9 | aacctctctc | 38 |
| hsa-miR-122a | UGGAGUGUGACAAUGGUGUUUGU | 39 |
| Lig. Temp. 10A | gtcagttgtcacaaacaccattgtcacactccactctcaggtt | 40 |
| Lig. Temp. 10B | tcatcacacgacaaacaccattgtcacactccactctcaggtt | 41 |
| Immob. Oligo 10 | aacctgagag | 42 |
| hsa-miR-124a | UUAAGGCACGCGGUGAAUGCCA | 43 |
| Lig. Temp. 11A | gtcagttgtctggcattcaccgcgtgccttaagggtatcgtt | 44 |
| Lig. Temp. 11B | tcatcacacgtggcattcaccgcgtgccttaagggtatcgtt | 45 |
| Immob. Oligo 11 | aaaaaaaaaaaacgataccc | 46 |
| hsa-miR-125a | UCCCUGAGACCCUUUAACCUGUG | 47 |
| Lig. Temp. 12A | gtcagttgtccacaggttaaagggtctcagggatggcctagtt | 48 |
| Lig. Temp. 12B | tcatcacacgcacaggttaaagggtctcagggatggcctagtt | 49 |
| Immob. Oligo 12 | aaaaaaaaaaaactaggcca | 50 |
| hsa-miR-129 | CUUUUUGCGGUCUGGGCUUGC | 51 |
| Lig. Temp. 13A | gtcagttgtcgcaagcccagaccgcaaaaagagggagagtt | 52 |
| Lig. Temp. 13B | tcatcacacggcaagcccagaccgcaaaaagagggagagtt | 53 |
| Immob. Oligo 13 | aaaaaaaaaaaactctccct | 54 |
| hsa-miR-130a | CAGUGCAAUGUUAAAAGGGCAU | 55 |
| Lig. Temp. 14A | gtcagttgtcatgcccttttaacattgcactgcctcagtctt | 56 |
| Lig. Temp. 14B | tcatcacacgatgcccttttaacattgcactgcctcagtctt | 57 |
| Immob. Oligo 14 | aaaaaaaaaaaagactgagg | 58 |
| hsa-miR-143 | UGAGAUGAAGCACUGUAGCUCA | 59 |
| Lig. Temp. 15A | gtcagttgtctgagctacagtgcttcatctcagcttgctctt | 60 |
| Lig. Temp. 15B | tcatcacacgtgagctacagtgcttcatctcagcttgctctt | 61 |
| Immob. Oligo 15 | aaaaaaaaaaaagagcaagc | 62 |
| hsa-miR-155 | UUAAUGCUAAUCGUGAUAGGGG | 63 |
| Lig. Temp. 16A | gtcagttgtccccctatcacgattagcattaactgactgctt | 64 |
| Lig. Temp. 16B | tcatcacacgcccctatcacgattagcattaactgactgctt | 65 |
| Immob. Oligo 16 | aaaaaaaaaaaagcagtcag | 66 |
| hsa-miR-183 | UAUGGCACUGGUAGAAUUCACUG | 67 |
| Lig. Temp. 17A | gtcagttgtccagtgaattctaccagtgccatatgttccgctt | 68 |
| Lig. Temp. 17B | tcatcacacgcagtgaattctaccagtgccatatgttccgctt | 69 |
| Immob. Oligo 17 | aaaaaaaaaaaagcggaaca | 70 |
| hsa-miR-185 | UGGAGAGAAAGGCAGUUC | 71 |
| Lig. Temp. 18A | gtcagttgtcgaactgcctttctctccagtgcttcctt | 72 |
| Lig. Temp. 18B | tcatcacacggaactgcctttctctccagtgcttcctt | 73 |
| Immob. Oligo 18 | aaaaaaaaaaaaggaagcac | 74 |
| hsa-miR-193a | AACUGGCCUACAAAGUCCCAG | 75 |
| Lig. Temp. 19A | gtcagttgtcctgggactttgtaggccagttggtactcctt | 76 |
| Lig. Temp. 19B | tcatcacacgctgggactttgtaggccagttggtactcctt | 77 |
| Immob. Oligo 19 | aaaaaaaaaaaaggagtacc | 78 |
| hsa-miR-198 | GGUCCAGAGGGGAGAUAGG | 79 |
| Lig. Temp. 20A | gtcagttgtccctatctcccctctggaccctatggcctt | 80 |
| Lig. Temp. 20B | tcatcacacgcctatctcccctctggaccctatggcctt | 81 |
| Immob. Oligo 20 | aaaaaaaaaaaaggccatag | 82 |
| hsa-miR-214 | ACAGCAGGCACAGACAGGCAG | 83 |
| Lig. Temp. 21A | gtcagttgtcctgcctgtctgtgcctgctgtaccaccactt | 84 |
| Lig. Temp. 21B | tcatcacacgctgcctgtctgtgcctgctgtaccaccactt | 85 |
| Immob. Oligo 21 | aagtggtggU | 86 |
| hsa-miR-320 | AAAAGCUGGGUUGAGAGGGCGAA | 87 |
| Lig. Temp. 22A | gtcagttgtcttcgccctctcaacccagcttttcaaccggatt | 88 |
| Lig. Temp. 22B | tcatcacacgttcgccctctcaacccagcttttcaaccggatt | 89 |
| Immob. Oligo 22 | aatccggttG | 90 |
| hsa-miR-346 | UGUCUGCCCGCAUGCCUGCCUCU | 91 |
| Lig. Temp. 23A | gtcagttgtcagaggcaggcatgcgggcagacacaagggttgt | 92 |
| Lig. Temp. 23B | tcatcacacgagaggcaggcatgcgggcagacacaagggttgt | 93 |
| Immob. Oligo 23 | aaaaaaaaaaacaacccttG | 94 |
| hsa-miR-370 | GCCUGCUGGGGUGGAACCUGG | 95 |
| Lig. Temp. 24A | gtcagttgtcccaggttccaccccagcaggcgacaagctgt | 96 |
| Lig. Temp. 24B | tcatcacacgccaggttccaccccagcaggcgacaagctgt | 97 |
| Immob. Oligo 24 | aaaaaaaaaaacagcttgtC | 98 |

*Ribonucleotides are shown in uppercase, and deoxyribonucleotides are shown in lowercase.

(iii) RNA Preparation

The levels of the mature microRNAs were analyzed in two different human cell lines (i.e., 293T cells and A549 cells). Adherent cells of 293T (ATCC Number: CRL-11269) were grown to about 80% confluency in DMEM medium (Product Number D6171; Sigma-Aldrich, St. Louis, Mo.), supplemented with 10% FBS, 8 mM L-glutamine, and 1 mM sodium pyruvate. Adherent cells of A549 (ATCC Number: CCL-185) were also grown to about 80% confluency in Mixture F12 medium (Product No. N4888; Sigma-Aldrich), supplemented with 10% FBS and 4 mM L-glutamine. Small RNA was isolated from each of the two cell lines with a small RNA purification kit (Product Number: SNC-50; Sigma-Aldrich) according to the kit's instructions. An analysis of the isolated RNA samples by a microfluidics-based system (Bioanalyzer; Agilent Technologies, Santa Clara, Calif.) showed that each RNA sample comprised overwhelmingly small ribosomal RNAs and tRNAs. A small fraction of each sample comprised microRNAs, but they were detectable only by PCR analysis.

(iv) Ligation of Mature microRNAs to Immobilized Oligonucleotides

Two experiments were conducted to compare the relative expression levels of the 24 mature microRNAs between these two cell lines. The first experiment comprised ligating the microRNAs to immobilized oligonucleotides at 35° C., and the second experiment comprised ligating the microRNAs to immobilized oligonucleotides at 37° C. Each experiment comprised two additional permutations. In the first permutation, an aliquot of 293T RNA was combined with the first set of ligation templates (Set A) and the Cy3 detection tag, and an aliquot of A549 RNA was combined with the second set of ligation templates (Set B) and the Cy5 detection tag, and the two samples were then mixed together after the detection tag ligation. In the second permutation, an aliquot of 293T RNA was combined with the second set of ligation templates (Set B) and the Cy5 detection tag, and an aliquot of A549 RNA was combined with the first set of ligation templates (Set A) and the Cy3 detection tag, and the two samples were then mixed together after the detection tag ligation. Control reactions were carried out concomitantly, except that T4 DNA ligase was omitted from the reactions.

For each reaction, an RNA sample (100 ng) was first combined with a ligation template pool in a 6-μl reaction, comprising 10 fmoles of each ligation template and 10 mM Tris-HCl (pH 7.6). The reaction was incubated in a thermocycler with a temperature gradient comprising 90° C. for 2 minutes, 60° C. for 10 minutes, 55° C. for 30 minutes, 50° C. for 30 minutes, and 45° C. for 10 minutes. The reaction was then brought up to 10 μl with 1× ligation buffer comprising 250 fmoles of a detection tag, 5% PEG 4000, and 10 Weiss units of T4 DNA ligase. The ligase was omitted from each control reaction. The detection tag ligation was conducted at 37° C. for 3 hours in a thermocyler. The ligation buffer (10×) comprised 400 mM Tris-HCl (pH 7.6), 100 mM MgCl₂, 1 mM ATP, and 1 mM DTT. Following the detection tag ligation, the 293T/Cy3 reaction was combined with the A549/Cy5 reaction, and the 293T/Cy5 reaction was combined with the A549/Cy3 reaction.

Each combined sample was further brought up to 70 μl with 1× ligation buffer comprising 5% PEG 4000, 0.1 μg/μl BSA, 0.15 μg/μl sodium polyglutamic acid (MW 15,000-50,000, Product Number P4761; Sigma-Aldrich), 0.1% Triton X-100, and 45 Weiss units of T4 DNA ligase. The ligation buffer was as described above. The ligase was again omitted from each control reaction. Each combined sample was loaded onto a separate section of a 4-array gasket slide (Agilent Technologies), and a slide that comprised 4 arrays of the immobilized oligonucleotides on the corresponding sections was then attached to the gasket slide. A microarray hybridization chamber (Agilent Technologies) was used to clamp the slides together to form four sealed chambers, each containing a 70-μl reaction. The slide assembly was immediately placed in a hybridization oven (Agilent Technologies). The array ligation was carried out either at 35° C. or at 37° C. for 16 hours. The ligation samples were mixed by rotation at 20 rpm during the incubation period.

After the array ligation, each slide was first washed in 0.5% SDS at 70° C. for 15 minutes, rinsed with 70° C. deionized water, and then further washed in deionized water at 70° C. for 15 minutes. Each slide was plunged up and down several times during each wash step. After a final rinse with 70° C. deionized water, each slide was dried with N₂ gas and scanned immediately on a microarray scanner (The ScanArray Express; Perkin Elmer, Waltham, Mass.) with 10 μm resolution and 90% laser power. The Cy5 channel was scanned with 75% PMT and the Cy3 channel was scanned with 85% PMT. Data were analyzed with ScanArray Express Software.

The results for the ligation at 35° C. are summarized in Table 2, and the results for the ligation at 37° C. are summarized in Table 3. In both experiments, no spots were detectable in the arrays that were contacted with the control reactions that contained no T4 DNA ligase. This finding revealed that hybridization alone did not contribute to the detection of the target microRNAs. In the arrays that were contacted with the reaction mixtures comprising T4 DNA ligase, target microRNAs were detectable on both the short (10 nucleotides) and the long (20 nucleotides) immobilized oligonucleotides. There was no correlation between the intensity of the fluorescent signal and the length of the immobilized oligonucleotide, however. Furthermore, target microRNAs were also detected on the immobilized oligonucleotides that were modified with a ribonucleotide at the 3′ end. This finding revealed that T4 DNA ligase was active in ligating the 5′ terminal phosphate group of an RNA molecule to the 3′ hydroxyl group of a DNA molecule or an RNA molecule immobilized on a solid support, in the presence of a DNA molecule as template. Therefore, both a DNA and an RNA oligonucleotide may be used as an immobilized oligonucleotide according to the method of the invention.

The results further showed that the intensity of the fluorescent signal differed greatly from microRNA to microRNA and from cell line to cell line for certain microRNAs. Within these two experiments, the fluorescent intensity was generally higher when the array ligation was carried out at 35° C. than when the array ligation was carried out at 37° C. Without being bound by a particular theory, this difference suggests that the stability of the 10 base-pair duplex between an artificial sequence of an immobilized oligonucleotide and its complementary region of a ligation template may be less stable at 37° C. than at 35° C.

The fluorescent intensity data from each experiment was normalized by hsa-miR-16 for each dye and each array to account for the differences between the two cyanine dyes and differences between the two arrays in each experiment. It is known that hsa-miR-16 is expressed at an equivalent level among different tissues and cell lines and, therefore, is regarded as a “house-keeping” microRNA. Accordingly, hsa-miR-16 was expressed at nearly equivalent levels in the 293T and A549 cell lines (see Tables 2 and 3). Normalization of the data showed that the relative expression levels of the target microRNAs within these two cell lines were quite similar between these two experiments. The results further showed that most of the 24 microRNAs were expressed at similar levels in these two cell lines, since most of their normalized expression ratios were close to 1. Nevertheless, a few microRNAs were differentially expressed. For example, hsa-miR-23b and hsa-miR-21 were expressed at much higher levels in A549 cells than in 293T cells, and hsa-let-7a was also expressed at a higher level in A549 than in 293T cells. These three microRNAs were subsequently analyzed by a SYBR® Green real-time qPCR method and the results also showed that hsa-miR-21 and hsa-miR-23b were expressed at much higher levels in A549 cells than in 293T cells. The qPCR analysis also showed a higher level of hsa-let-7a in A549 cells than in 293T cells, although the difference was smaller in the qPCR analysis.

Taken together, this example demonstrated that a template-dependent ligase is essential for capturing target microRNAs to immobilized oligonucleotides according to the method of the invention. It also showed that the 5′ terminal phosphate group of an RNA molecule may be ligated to the 3′ hydroxyl group of an oligonucleotide immobilized on a solid support, in the presence of a template-dependent DNA ligase and a DNA molecule as template. This example further illustrated that two populations of target microRNAs may be simultaneously analyzed on an array of immobilized oligonucleotides by the method of the invention.

TABLE 2
Ligation Array Analysis Using Immobilized Oligonucleotides Having Free 3′
OH Groups and 35° C. Ligation.

| | | Average of Mean Fluorescent | | | | Normalized |
|---|---|---|---|---|---|---|
| | SEQ | Intensity Minus Background | | | | Expression |
| | ID | Array Number 1 | | Array Number 2 | | Ratios |
| MicroRNA | NO: | 293T/Cy3 | A549/Cy5 | 293T/Cy5 | A549/Cy3 | of 293T/A549 |
| hsa-let-7a | 3 | 2805 | 13306 | 2904 | 8825 | 0.22 |
| hsa-miR-16 | 7 | 5149 | 5969 | 5423 | 3124 | 1.00 |
| hsa-miR-21 | 11 | 1040 | 55386 | 1808 | 16012 | 0.04 |
| hsa-miR-23b | 15 | 464 | 11361 | 818 | 5601 | 0.07 |
| hsa-miR-29a | 19 | 554 | 2061 | 420 | 520 | 0.39 |
| hsa-miR-29b | 23 | 254 | 306 | 418 | 471 | 0.74 |
| hsa-miR-30b | 27 | 1595 | 994 | 680 | 1368 | 1.07 |
| hsa-miR-31 | 31 | 297 | 401 | 288 | 134 | 1.05 |
| hsa-miR-34a | 35 | 224 | 947 | 601 | 148 | 1.31 |
| hsa-miR-122a | 39 | 967 | 1532 | 787 | 391 | 0.95 |
| hsa-miR-124a | 43 | 304 | 623 | 475 | 182 | 1.03 |
| hsa-miR-125a | 47 | 396 | 1190 | 535 | 270 | 0.76 |
| hsa-miR-129 | 51 | 325 | 858 | 447 | 78 | 1.86 |
| hsa-miR-130a | 55 | 1361 | 1995 | 1716 | 787 | 1.02 |
| hsa-miR-143 | 59 | 245 | 2397 | 1454 | 167 | 2.57 |
| hsa-miR-155 | 63 | 16639 | 5226 | 2634 | 11161 | 1.91 |
| hsa-miR-183 | 67 | 210 | 297 | 288 | 100 | 1.24 |
| hsa-miR-185 | 71 | 5391 | 22441 | 14099 | 4080 | 1.13 |
| hsa-miR-193a | 75 | 2804 | 3073 | 2087 | 2226 | 0.80 |
| hsa-miR-198 | 79 | 2219 | 2651 | 1108 | 829 | 0.87 |
| hsa-miR-214 | 83 | 1451 | 2688 | 2531 | 1296 | 0.88 |
| hsa-miR-320 | 87 | 668 | 1337 | 899 | 374 | 0.98 |
| hsa-miR-346 | 91 | 969 | 1509 | 1508 | 770 | 0.94 |
| hsa-miR-370 | 95 | 35 | 109 | 97 | 22 | 1.46 |

TABLE 3
Ligation Array Analysis Using Immobilized Oligonucleotides Having Free 3′
OH Groups and 37° C. Ligation.

| | | Average of Mean Fluorescent | | | | Normalized |
|---|---|---|---|---|---|---|
| | SEQ | Intensity Minus Background | | | | Expression |
| | ID | Array Number 1 | | Array Number 2 | | Ratios |
| MicroRNA | NO: | 293T/Cy3 | A549/Cy5 | 293T/Cy5 | A549/Cy3 | of 293T/A549 |
| hsa-let-7a | 3 | 2401 | 6954 | 1010 | 3904 | 0.24 |
| hsa-miR-16 | 7 | 3730 | 2704 | 2004 | 1751 | 1.00 |
| hsa-miR-21 | 11 | 726 | 28748 | 457 | 4257 | 0.06 |
| hsa-miR-23b | 15 | 603 | 6151 | 618 | 4550 | 0.09 |
| hsa-miR-29a | 19 | 559 | 1285 | 147 | 184 | 0.51 |
| hsa-miR-29b | 23 | 263 | 562 | 366 | 366 | 0.61 |
| hsa-miR-30b | 27 | 446 | 232 | 197 | 273 | 1.01 |
| hsa-miR-31 | 31 | 185 | 148 | 156 | 89 | 1.21 |
| hsa-miR-34a | 35 | 111 | 268 | 249 | 103 | 1.21 |
| hsa-miR-122a | 39 | 411 | 412 | 290 | 121 | 1.41 |
| hsa-miR-124a | 43 | 115 | 136 | 236 | 120 | 1.17 |
| hsa-miR-125a | 47 | 229 | 376 | 292 | 127 | 1.23 |
| hsa-miR-129 | 51 | 257 | 508 | 229 | 63 | 1.77 |
| hsa-miR-130a | 55 | 730 | 1401 | 1061 | 583 | 0.98 |
| hsa-miR-143 | 59 | 377 | 1952 | 374 | 79 | 2.14 |
| hsa-miR-155 | 63 | 13634 | 2758 | 2264 | 10294 | 1.89 |
| hsa-miR-183 | 67 | 217 | 199 | 195 | 93 | 1.31 |
| hsa-miR-185 | 71 | 3552 | 8630 | 5705 | 1584 | 1.72 |
| hsa-miR-193a | 75 | 739 | 573 | 415 | 424 | 0.90 |
| hsa-miR-198 | 79 | 641 | 468 | 278 | 179 | 1.17 |
| hsa-miR-214 | 83 | 376 | 337 | 318 | 151 | 1.32 |
| hsa-miR-320 | 87 | 367 | 303 | 299 | 220 | 1.03 |
| hsa-miR-346 | 91 | 346 | 297 | 282 | 172 | 1.14 |
| hsa-miR-370 | 95 | 129 | 119 | 102 | 68 | 1.05 |

### Example 2. Ligation Array Analyses Using Immobilized Oligonucleotides with 5′ Terminal Phosphate Groups

The following example was designed to determine whether the 3′ terminal hydroxyl group of an RNA molecule may be ligated to the 5′ terminal phosphate group of an oligonucleotide immobilized on a solid support via the catalytic activity of a template-dependent ligase in the presence of a ligation template, as depicted in FIG. 2.

(i) Immobilized Oligonucleotides Ligation Templates and Detection Tags

All oligonucleotides used in this example were synthesized by conventional techniques. Each oligonucleotide for immobilization onto glass slides was 20 nucleotides in length. That is, each comprised a unique artificial sequence of 10 nucleotides, with 50% GC content, and an extension of 10 As at the 3′ end (see Table 4). Each oligonucleotide was modified with a C6-amine group at the 3′ end and a phosphate group at the 5′ end during synthesis. Each oligonucleotide was immobilized via its 3′ end onto CodeLink glass slides in five locations. The oligonucleotide immobilization and post immobilization slide treatment procedures were as described in Example 1.

Two sets of ligation templates were synthesized for analyzing 11 human mature microRNAs in 293T and A549 adherent cells. Each of the 11 microRNAs was analyzed by a pair of ligation templates that differed only in the region that was complementary to a detection tag. Each ligation template comprised (5′ to 3′) a first region (10 nucleotides in length) that was complementary to an artificial sequence of a particular immobilized oligonucleotide, a second region that was complementary to a human mature microRNA, and a third region (10 nucleotides in length) that was complementary to an oligonucleotide portion of a detection tag. The first set of ligation templates (Set A) comprised the third region that was complementary to a Cy3 detection tag and the second set of ligation templates (Set B) comprised the third region that was complementary to a Cy5 detection tag. The Cy3 detection tag comprised 5′-Cy3 -atagtcagtcaacaG (SEQ ID NO: 99), where G is a ribonucleotide. The Cy5 detection tag comprised 5′ -Cy5-atagtagtagtgtgC (SEQ ID NO: 100), where C is a ribonucleotide. The ligation templates in each set were combined to form a pool. The target microRNAs, ligation templates, and immobilized oligonucleotides are presented in Table 4.

TABLE 4

Target Mature MicroRNAs, Ligation Templates, and Immobilized
Oligonucleotides Used in Example 2.

| | | |
|---|---|---|
| Name | Sequence (5′ to 3′)* | SEQ ID NO: |

| | | |
|---|---|---|
| hsa-miR-16 | UAGCAGCACGUAAAUAUUGGCG | 7 |
| Lig. Temp. 25A | ccatgttggtcgccaatatttacgtgctgctactgttgactg | 101 |
| Lig. Temp. 25B | ccatgttggtcgccaatatttacgtgctgctagcacactact | 102 |
| Immob. Oligo 25 | accaacatggaaaaaaaaaa | 103 |
| hsa-let-7a | UGAGGUAGUAGGUUGUAUAGUU | 3 |
| Lig. Temp. 26A | gtgcattggtaactatacaacctactacctcactgttgactg | 104 |
| Lig. Temp. 26B | gtgcattggtaactatacaacctactacctcagcacactact | 105 |
| Immob. Oligo 26 | accaatgcactaaaaaaaaa | 106 |
| hsa-miR-21 | UAGCUUAUCAGACUGAUGUUGA | 11 |
| Lig. Temp. 27A | cttaggtggttcaacatcagtctgataagctactgttgactg | 107 |
| Lig. Temp. 27B | cttaggtggttcaacatcagtctgataagctagcacactact | 108 |
| Immob. Oligo 27 | accacctaaggaaaaaaaaa | 109 |
| hsa-miR-23b | AUCACAUUGCCAGGGAUUACC | 15 |
| Lig. Temp. 28A | atcatgcggtggtaatccctggcaatgtgatctgttgactg | 110 |
| Lig. Temp. 28B | atcatgcggtggtaatccctggcaatgtgatgcacactact | 111 |
| Immob. Oligo 28 | accgcatgatcaaaaaaaaa | 112 |
| hsa-miR-29a | UAGCACCAUCUGAAAUCGGUU | 19 |
| Lig. Temp. 29A | Tcgtcatcgtaaccgatttcagatggtgctactgttgactg | 113 |
| Lig. Temp. 29B | Tcgtcatcgtaaccgatttcagatggtgctagcacactact | 114 |
| Immob. Oligo 29 | acgatgacgaataaaaaaaa | 115 |
| hsa-miR-29b | UAGCACCAUUUGAAAUCAGUGUU | 23 |
| Lig. Temp. 30A | agtcttgcgtaacactgatttcaaatggtgctactgttgactg | 116 |
| Lig. Temp. 30B | agtcttgcgtaacactgatttcaaatggtgctagcacactact | 117 |
| Immob. Oligo 30 | acgcaagactagaaaaaaaa | 118 |
| hsa-miR-30b | UGUAAACAUCCUACACUCAGCU | 27 |
| Lig. Temp. 31A | ataacggcgtagctgagtgtaggatgtttacactgttgactg | 119 |
| Lig. Temp. 31B | ataacggcgtagctgagtgtaggatgtttacagcacactact | 120 |
| Immob. Oligo 31 | acgccgttatacaaaaaaaa | 121 |
| hsa-miR-31 | GGCAAGAUGCUGGCAUAGCUG | 31 |
| Lig. Temp. 32A | tatcaggcgtcagctatgccagcatcttgccctgttgactg | 122 |
| Lig. Temp. 32B | tatcaggcgtcagctatgccagcatcttgccgcacactact | 123 |
| Immob. Oligo 32 | acgcctgatagtaaaaaaaa | 124 |
| hsa-miR-34a | UGGCAGUGUCUUAGCUGGUUGUU | 35 |
| Lig. Temp. 33A | tcaagtccgtaacaaccagctaagacactgccactgttgactg | 125 |
| Lig. Temp. 33B | tcaagtccgtaacaaccagctaagacactgccagcacactact | 126 |
| Immob. Oligo 33 | acggacttgactaaaaaaaa | 127 |
| hsa-miR-122a | UGGAGUGUGACAAUGGUGUUUGU | 39 |
| Lig. Temp. 34A | ttagagccgtacaaacaccattgtcacactccactgttgactg | 128 |
| Lig. Temp. 34B | ttagagccgtacaaacaccattgtcacactccagcacactact | 129 |
| Immob. Oligo 34 | acggctctaatgaaaaaaaa | 130 |
| hsa-miR-124a | UUAAGGCACGCGGUGAAUGCCA | 43 |
| Lig. Temp. 35A | ggtaagacgttggcattcaccgcgtgccttaactgttgactg | 131 |
| Lig. Temp. 35B | ggtaagacgttggcattcaccgcgtgccttaagcacactact | 132 |
| Immob. Oligo 35 | acgtcttacctcaaaaaaaa | 133 |

*Ribonucleotides are shown in uppercase, and deoxyribonucleotides are shown in lowercase.

(ii) RNA Preparation

Small RNA was prepared from 293T and A549 adherent cells, respectively, as described in Example 1. As in Example 1, the RNA from the different cells was mixed with different ligation templates and different detection tags. In the first permutation, an aliquot of 293T RNA was combined with the first set of ligation templates (Set A) and the Cy3 detection tag, and an aliquot of A549 RNA was combined with the second set of ligation templates (Set B) and the Cy5 detection tag. The two samples were then mixed together after the detection tag ligation. In the second permutation, an aliquot of 293T RNA was combined with the second set of ligation templates (Set B) and the Cy5 detection tag, and an aliquot of A549 RNA was combined with the first set of ligation templates (Set A) and the Cy3 detection tag. The two samples were then mixed together after the detection tag ligation. Control reactions were carried out concomitantly, except that T4 DNA ligase was omitted from the reactions.

(iii) Ligation Reaction

For each reaction, an RNA sample (100 ng) was first combined with a ligation template pool in a 6-μl reaction, comprising 10 fmoles of each ligation template and 10 mM Tris-HCl (pH 7.6). The reaction was incubated in a thermocycler with a temperature gradient comprising 90° C. for 2 minutes, 60° C. for 10 minutes, 55° C. for 30 minutes, 50° C. for 30 minutes, and 45° C. for 10 minutes. The reaction was then brought up to 10 μl with 1× ligation buffer comprising 50 fmoles of a detection tag, 5% PEG 4000, and 10 Weiss units of T4 DNA ligase. The detection tag ligation was conducted at 37° C. for 3 hours in a thermocyler. The ligation buffer was as described in Example 1. T4 DNA ligase was omitted from the control reactions. Following the detection tag ligation, the 293T/Cy3 reaction was combined with the A549/Cy5 reaction, and the 293T/Cy5 reaction was combined with the A549/Cy3 reaction.

Each combined sample was further brought up to 70 μl with 1× ligation buffer comprising 5% PEG 4000, 0.1 μg/μl BSA, 0.15 μg/μl sodium polyglutamic acid, 0.1% Triton X-100, and 45 Weiss units of T4 DNA ligase. The ligation buffer was as described in Example 1. T4 DNA ligase was omitted from the control reactions. Each sample was applied to a separate section of a 4-array gasket slide (Agilent Technologies), and a slide that comprised 4 arrays of the immobilized oligonucleotides on the corresponding sections was then attached to the gasket slide as described in Example 1. Array ligation was carried out in a hybridization oven (Agilent Technologies) at 35° C. with 20 rpm for 16 hours. The post-ligation slide wash procedure was as described in Example 1. The Cy5 channel was scanned at 80% PMT and 90% laser power and the Cy3 channel was scanned at 85% PMT and 90% laser power. Fluorescent intensity data was normalized by hsa-miR-16 as described in Example 1.

The results were summarized in Table 5. No spots were detectable in the arrays that were contacted with the control reactions that contained no T4 DNA ligase, again showing that a template-dependent ligase is essential for capturing target microRNAs to the immobilized oligonucleotides. In the arrays that were contacted with the reactions that contained T4 DNA ligase, fluorescent signals were detected indicating ligation of the microRNAs to the immobilized oligonucleotides. The intensity of the fluorescent signals differed greatly from microRNA to microRNA and from cell line to cell line for some microRNAs. Furthermore, after data normalization by hsa-miR-16, the expression ratios of the 11 microRNAs for these two cell lines were generally consistent with the results of Example 1. For example, as was observed in Example 1, the three microRNAs, hsa-let-7a, hsa-miR-23b, and hsa-miR-21, were also expressed at a higher level in A549 cells than in 293T cells, although the difference for hsa-miR-23b was smaller in this experiment.

This example demonstrated that the 3′ hydroxyl group of an RNA molecule may be ligated to the 5′ terminal phosphate group of an oligonucleotide immobilized on a solid support in the presence of a template-dependent ligase and a template DNA molecule. This example further illustrated that two populations of microRNAs may be simultaneously analyzed on an array of immobilized oligonucleotides having 5′ terminal phosphate groups according to the method of the invention.

TABLE 5
Ligation Array Analysis Using Immobilized Oligonucleotides Having 5′
Phosphate Groups

| | |
|---|---|
| Average of Mean Fluorescent | Normalized |
| Intensity minus Background | Expression |

| | | | |
|---|---|---|---|
| SEQ | Array Number 1 | Array Number 2 | Ratios of |

| MicroRNA | ID NO: | 293T/Cy3 | A549/Cy5 | 293T/Cy5 | A549/Cy3 | 293T/A549 |
|---|---|---|---|---|---|---|
| hsa-let-7a | 3 | 1419 | 2543 | 811 | 3883 | 0.28 |
| hsa-miR-16 | 7 | 5720 | 3851 | 3003 | 2517 | 1.00 |
| hsa-miR-21 | 11 | 142 | 9966 | 79 | 447 | 0.08 |
| hsa-miR-23b | 15 | 1658 | 2537 | 1328 | 2832 | 0.42 |
| hsa-miR-29a | 19 | 196 | 428 | 226 | 1608 | 0.21 |
| hsa-miR-29b | 23 | 115 | 306 | 145 | 313 | 0.32 |
| hsa-miR-30b | 27 | 249 | 190 | 122 | 108 | 0.92 |
| hsa-miR-31 | 31 | 329 | 361 | 277 | 390 | 0.60 |
| hsa-miR-34a | 35 | 185 | 376 | 190 | 55 | 1.61 |
| hsa-miR-122a | 39 | 27 | 105 | 90 | 36 | 1.13 |
| hsa-miR-124a | 43 | 115 | 93 | 74 | 85 | 0.78 |

### Example 3. Ligating Target MicroRNA to Immobilized Oligonucleotides at a Higher Temperature Using a Thermophilic DNA Ligase

The purpose of this example was to evaluate whether a template-dependent thermophilic DNA ligase could be used for high temperature ligation of an RNA molecule to a DNA molecule immobilized on a solid support in the presence of a template DNA molecule. Homogeneous solution ligation analyses revealed that both Taq DNA ligase and 9° N DNA ligase were active in ligating the 3′-hydroxyl group of an RNA molecule to the 5′ terminal phosphate group of a DNA molecule in the presence of a template DNA molecule. It is well known that Taq DNA ligase is active between 45° C. and 65° C., and that 9° N DNA ligase is active between 45° C. and 90° C. Since the later is active at a wider range of temperatures, this ligase was used in this experiment. The ligase was tested with different lengths of base pairing between an immobilized oligonucleotide and its complementary counterpart on a ligation template.

(i) Oligonucleotides

All oligonucleotides used in this experiment were synthesized by conventional techniques. Each oligonucleotide for immobilization onto CodeLink slides was 20 nucleotides in length (see Table 6) and was modified with a C6-amine at the 3′ end and a phosphate group at the 5′ end during synthesis. Each oligonucleotide was immobilized (via its 3′ end) onto CodeLink slides in five locations in each array, and each slide comprised four arrays. The oligonucleotide immobilization and post-immobilization slide treatment procedures were as described in Example 1. Each ligation template comprised (5′ to 3′) a first region that was complementary to a unique artificial sequence of a particular immobilized oligonucleotide, a second region that was complementary to hsa-miR-16 microRNA (SEQ ID NO: 7), and a third region that was complementary to the Cy3 detection tag (SEQ ID NO: 99) that was described in Example 2. The length of the first region differed from template to template. In a first test, the length of the first region ranged from 14 to 18 nucleotides, with a GC content ranging from 43% and 50%. In a subsequent second test, the length of the first region ranged from 10 to 16 nucleotides, with a GC content ranging from 42% to 50%. The target microRNA, immobilized oligonucleotides, and ligation templates are presented in Table 6.

TABLE 6

Target MicroRNA, Ligation Templates, and Immobilized Oligonucleotides
Used in Example 3.

| | | |
|---|---|---|
| | | SEQ ID |
| Name | Sequence (5′-3′)* | NO: |

First Test

| | | |
|---|---|---|
| hsa-miR-16 | UAGCAGCACGUAAAUAUUGGCG | 7 |
| Lig. Temp. 36 | aacaaacgctacacgtctcgccaatatttacgtgctgctactgttgactgac | 134 |
| Immob. Oligo 36 | agacgtgtagcgtttgtttc | 135 |
| hsa-miR-16 | UAGCAGCACGUAAAUAUUGGCG | 7 |
| Lig. Temp. 37 | ggtttgacgacctagtcgccaatatttacgtgctgctactgttgactgac | 136 |
| Immob. Oligo 37 | actaggtcgtcaaaccaaga | 137 |
| hsa-miR-16 | UAGCAGCACGUAAAUAUUGGCG | 7 |
| Lig. Temp. 38 | ttctgacccttagtcgccaatatttacgtgctgctactgttgactgac | 138 |
| Immob. Oligo 38 | actaagggtcagaaatgcca | 139 |

Second Test

| | | |
|---|---|---|
| hsa-miR-16 | UAGCAGCACGUAAAUAUUGGCG | 7 |
| Lig. Temp. 37 | ggtttgacgacctagtcgccaatatttacgtgctgctactgttgactgac | 136 |
| Immob. Oligo 37 | actaggtcgtcaaaccaaga | 137 |
| hsa-miR-16 | UAGCAGCACGUAAAUAUUGGCG | 7 |
| Lig. Temp. 38 | ttctgacccttagtcgccaatatttacgtgctgctactgttgactgac | 138 |
| Immob. Oligo 38 | actaagggtcagaaatgcca | 139 |
| hsa-miR-16 | UAGCAGCACGUAAAUAUUGGCG | 7 |
| Lig. Temp. 39 | attcgtcatcgtcgccaatatttacgtgctgctactgttgactgac | 140 |
| Immob. Oligo 29 | acgatgacgaataaaaaaaa | 115 |
| hsa-miR-16 | UAGCAGCACGUAAAUAUUGGCG | 7 |
| Lig. Temp. 40 | cttaggtggtcgccaatatttacgtgctgctactgttgactg | 141 |
| Immob. Oligo 27 | accacctaaggaaaaaaaaa | 109 |

*Ribonucleotides are shown in uppercase, and deoxyribonucleotides are shown in lowercase.

(ii) Ligation Reaction

Small RNA was prepared from 293T adherent cells as described in Example 1. For each reaction, an aliquot (200 ng) of 293T RNA was combined with a ligation template in a 6-μl reaction, comprising 10 fmoles of a ligation template and 10 mM Tris-HCl (pH 7.6). The reaction was incubated in a thermocycler with a temperature gradient comprising 90° C. for 2 minutes, 60° C. for 10 minutes, 55° C. for 30 minutes, 50° C. for 30 minutes, and 45° C. for 10 minutes. Each reaction was then brought up to 10 μl with 1× ligation buffer comprising 50 fmoles of the Cy3 detection tag, 5% PEG 4000, and 10 Weiss units of T4 DNA ligase. The detection tag ligation reaction was conducted at 37° C. for 3 hours in a thermocyler, and then heated at 45° C. for 15 minutes to inactivate T4 DNA ligase. Each reaction was then brought up to 70 μl with 1× ligation buffer comprising 5% PEG 4000, 0.2 μg/μl BSA, 0.05% Triton X-100, and 100 units of 9° N DNA ligase. The 9° N DNA ligase was omitted from a control reaction, which comprised the ligation template with the longest first region. The ligation buffer was as described in Example 1. Each reaction was then loaded on to a section of a 4-array gasket slide and a slide comprising 4 arrays of the immobilized oligonucleotides was then attached to the gasket slide to form 4 sealed chambers, as described in Example 1. Array ligation was conducted at 45° C. and 20 rpm for 18 hours in a hybridization oven (Agilent Technologies). Each slide was first washed in 0.5% SDS at 70° C. for 15 minutes, rinsed with 70° C. deionized water, and then washed in deionized water at 70° C. for 10 minutes. Each slide was plunged up and down several times during each wash step. After a final rinse with 70° C. deionized water, the slide was dried with N₂ gas and scanned in the Cy3 channel at 10 μm resolution with 90% laser power and 85% PMT. The data were analyzed with ScanArray Express Software.

The results are summarized in Table 7. No spots were detectable in the array that was contacted with the control reaction that contained no 9° N DNA ligase. This showed that hybridization alone did not contribute to the fluorescent signal of hsa-miR-16 microRNA, even with the longest duplex (18 bp) between the ligation template and the immobilized oligonucleotide. In the arrays that were contacted with the reaction mixtures that contained 9° N DNA ligase, fluorescent signals were detected for all duplex lengths (i.e., from 10 to 18 bp). The intensity of the fluorescent signal decreased as the length of the duplex region decreased, however. The reduction in the fluorescent signal intensity was the most dramatic when the length of the duplex decreased from 12 to 10 bp. This suggests that the thermophilic DNA ligase requires at least 12 bp of duplex at one side of the ligation juncture for efficient ligation. There was virtually no difference in the fluorescent signal intensity between 16 and 18 bp duplexes. This suggests that a small difference in the GC content may have also affected the ligation efficiency or that al 6 bp duplex with 50% GC was sufficiently stable at 45° C. for maximum ligation efficiency by the ligase under the experimental conditions.

Taken together, this example demonstrated that a thermophilic template-dependent DNA ligase may be used to ligate the 3′ hydroxyl group of an RNA molecule to the 5′ terminal phosphate group of a DNA molecule immobilized on a solid support in the presence of a template DNA molecule according to the method of the invention. This example further illustrated that, by using a thermophilic template-dependent DNA ligase, the length of the unique artificial sequence of an immobilized oligonucleotide may be proportionally increased in accordance with the increase in ligation temperature to achieve a desirable specificity and sensitivity in target detection and quantitation. Furthermore, increasing the length of the unique artificial sequence of an immobilized oligonucleotide will increase the repertoire of artificial sequences that are sufficiently different from one another. Therefore, the capacity of a universal ligation array may be effectively augmented by increasing the length of each artificial sequence and by using a template-dependent thermophilic ligase for high temperature ligation according to the method of the invention.

TABLE 7
Mean Fluorescent Intensity Decreases as the Number of Base Pairs
Between a Ligation Template and an Immobilized Oligonucleotide Decreases.

| | | | | | | |
|---|---|---|---|---|---|---|
| | | | | Base Pair and | | Mean Cy3 |
| | | | SEQ | % GC between | 9° N | Intensity of |
| | SEQ | | ID | Immob. Oligo | DNA | hsa-miR-16 |
| Name | ID NO: | Name | NO: | and Lig. Temp. | Ligase | (Average ± SD) |

Test 1

| | | | | | | |
|---|---|---|---|---|---|---|
| Lig. Temp. | 134 | Immob. | 135 | 18/44% | No | Not detectable |
| 36 | | Oligo 36 | | | | |
| Lig. Temp. | 134 | Immob. | 135 | 18/44% | Yes | 5653 ± 953 |
| 36 | | Oligo 36 | | | | |
| Lig. Temp. | 136 | Immob. | 137 | 16/50% | Yes | 5576 ± 469 |
| 37 | | Oligo 37 | | | | |
| Lig. Temp. | 138 | Immob. | 139 | 14/43% | Yes | 4170 ± 638 |
| 38 | | Oligo 38 | | | | |

Test 2

| | | | | | | |
|---|---|---|---|---|---|---|
| Lig. Temp. | 136 | Immob. | 137 | 16/50% | Yes | 3309 ± 590 |
| 37 | | Oligo 37 | | | | |
| Lig. Temp. | 138 | Immob. | 139 | 14/43% | Yes | 2769 ± 625 |
| 38 | | Oligo 38 | | | | |
| Lig. Temp. | 140 | Immob. | 115 | 12/42% | Yes | 1983 ± 165 |
| 39 | | Oligo 29 | | | | |
| Lig. Temp. | 141 | Immob. | 109 | 10/50% | Yes | 329 ± 93 |
| 40 | | Oligo 27 | | | | |

What is claimed is:

1. An array system comprising:
(a) a plurality of immobilized oligonucleotides covalently attached to a solid support at a plurality of distinct array positions, each array position comprising at least one immobilized oligonucleotide comprising a unique artificial sequence; and
(b) a plurality of ligation templates, each ligation template comprising a first region with complementarity to the unique artificial sequence of specific immobilized oligonucleotide and a second region with complementarity to all or part of a specific target nucleic acid, whereby each ligation template is able to direct a specific target nucleic acid to a specific immobilized oligonucleotide for subsequent ligation and detection.

2. The array of claim 1, wherein the unique artificial sequence of each immobilized oligonucleotide is from about 4 nucleotides to about 30 nucleotides in length and is located at the free end of the immobilized oligonucleotide.

3. The array of claim 1, wherein the solid support is a material selected from the group consisting of glasses, silicons, polymers, and metals; and the solid support has a form selected from the group consisting of a slide, a plate, a well, a microparticle, and a combination thereof.

4. The array of claim 3, wherein the solid support is further modified to contain a thin layer of three dimensional porous structures selected from the group consisting of a hydrophilic polymer gel, a dendrimer, and a combination thereof.

5. The array of claim 1, wherein each ligation template further comprises at least one molecule selected from the group consisting of a locked nucleic acid, biotin, and digoxigenin.

6. The array of claim 1, wherein each ligation template further comprises a region with complementarity to a portion of a detection tag.

7. The array of claim 1, wherein the target nucleic acid is selected from the group consisting of a mature small RNA molecule, a precursor small RNA molecule, a messenger RNA molecule, a cDNA molecule, a DNA molecule, and a fragment thereof.

8. A method for analyzing at least one population of nucleic acids, the method comprising:
(a) contacting an array of immobilized oligonucleotides with a plurality of target nucleic acids and a plurality of ligation templates, the immobilized oligonucleotides covalently attached to a solid support at a plurality of distinct array positions, each array position comprising at least one immobilized oligonucleotide comprising a unique artificial sequence, each ligation template comprising a first region with complementarity to the unique artificial sequence of a specific immobilized oligonucleotide and a second region with complementarity to all or part of a specific target nucleic acid, each target nucleic acid comprising a signaling means, wherein each target nucleic acid is directed to a specific immobilized oligonucleotide by a specific ligation template;
(b) ligating the plurality of target nucleic acids to the plurality of immobilized oligonucleotides in the presence of the plurality of ligation templates, thereby forming a plurality of ligation products, each ligation product comprising an immobilized oligonucleotide and a target nucleic acid having a signaling means, and
(c) quantifying the signal associated with each ligation product, thereby analyzing the population(s) of nucleic acids.

9. The method of claim 8, wherein the unique artificial sequence of each immobilized oligonucleotide is from about 4 nucleotides to about 30 nucleotides in length and is located at the free end of the immobilized oligonucleotide.

10. The method of claim 8, wherein the solid support is a material selected from the group consisting of glasses, silicons, polymers, and metals; and the solid support has a form selected from the group consisting of a slide, a plate, a well, a microparticle, and a combination thereof.

11. The method of claim 10, wherein the solid support is further modified to contain a thin layer of three dimensional porous structures selected from the group consisting of a hydrophilic polymer gel, a dendrimer, and a combination thereof.

12. The method of claim 8, wherein each ligation template further comprises at least one molecule selected from the group consisting of a locked nucleic acid, biotin, and digoxigenin.

13. The method of claim 8, wherein the ligation is catalyzed by a template-dependent ligase selected from the group consisting of T4 DNA ligase, T4 RNA ligase 2, vaccinia DNA ligase, E. coli DNA ligase, a mammalian DNA ligase, Taq DNA ligase, Tth DNA ligase, Tfi DNA ligase, Ampligase DNA ligase, 9° N DNA ligase, and a combination thereof.

14. The method of claim 8, wherein the population of nucleic acids that is analyzed and the plurality of target nucleic acids that is contacted with the array of immobilized oligonucleotides is a plurality of target mature small RNA molecules selected from the group consisting of mature microRNAs (miRNAs), mature short interfering RNAs (siRNAs), mature repeat associated siRNAs (rasiRNAs), mature transacting siRNAs (tasiRNAs), mature Piwi interacting RNAs (piRNAs), and mature 21-U RNAs.

15. The method of claim 14, wherein the signaling means of each target mature small RNA molecule comprises a detection tag that is ligated to the target mature small RNA molecule.

16. The method of claim 15, wherein the detection tag comprises an oligonucleotide portion and at least one signaling molecule, the oligonucleotide portion for ligating the detection tag to the target mature small RNA, the signaling molecule selected from the group consisting of a fluorescent dye, biotin, digoxigenin, and a sequence of nucleotides that is a target for branched DNA detection.

17. The method of claim 16, wherein each ligation template further comprises a region with complementarity to the oligonucleotide portion of the detection tag.

18. The method of claim 17, wherein the detection tag is ligated to the plurality of target mature small RNA molecules in the presence of the plurality of ligation templates, this ligation occurring prior to the ligation of the plurality of target mature small RNA molecules to the array of immobilized oligonucleotides.

19. The method of claim 17, wherein the detection tag is ligated to the plurality of target mature small RNA molecules in the presence of the plurality of ligation templates, this ligation occurring concurrently with the ligation of the plurality of target mature small RNA molecules to the array of immobilized oligonucleotides.

20. The method of claim 17, wherein each immobilized oligonucleotide has a free 3′ terminal hydroxyl group, each detection tag has a free 5′ terminal phosphate group, and the orientation of each ligation template is such that the 5′ end of a specific target mature small RNA molecule is ligated to a specific immobilized oligonucleotide and the 3′ end of the specific target mature small RNA molecule is ligated to the detection tag.

21. The method of claim 17, wherein each immobilized oligonucleotide has a free 5′ terminal phosphate group, each detection tag has a free 3′ terminal hydroxyl group, and the orientation of each ligation template is such that the 3′ end of a specific target mature small RNA molecule is ligated to a specific immobilized oligonucleotide and the 5′ end of the specific target mature small RNA molecule is ligated to the detection tag.

22. The method of claim 14, wherein the signaling means of each target mature small RNA molecule comprises at least one signaling molecule attached to the mature small RNA molecule, the signaling molecule selected from the group consisting of a fluorescent dye, biotin, and digoxigenin.

23. The method of claim 8, wherein the population of nucleic acids that is analyzed and the plurality of target nucleic acids that is contacted with the array of immobilized oligonucleotides is a plurality of target precursor small RNA molecules.

24. The method of claim 23, wherein the signaling means of each target precursor small RNA molecule comprises at least one signaling molecule attached to the precursor small RNA molecule, the signaling molecule selected from the group consisting of a fluorescent dye, biotin, and digoxigenin.

25. The method of claim 23, wherein each immobilized oligonucleotide has a free 3′ terminal hydroxyl group and the orientation of each ligation template is such that the 5′ end of the target precursor small RNA molecule is ligated to a specific immobilized oligonucleotide.

26. The method of claim 23, wherein each immobilized oligonucleotide has a free 5′ terminal phosphate group and the orientation of each ligation template is such that the 3′ end of the target precursor small RNA molecule is ligated to a specific immobilized oligonucleotide.

27. The method of claim 8, wherein the population of nucleic acids that is analyzed is a population of messenger RNA molecules, and the plurality of target nucleic acids that is contacted with the array of immobilized oligonucleotides is a plurality of target messenger RNA molecules or fragments thereof.

28. The method of claim 27, wherein the population of messenger RNA molecules is digested with an RNase H enzyme in the presence of a deoxyoligonucleotide template to give rise to the plurality of target messenger RNA fragments.

29. The method of claim 27, wherein the population of messenger RNA molecules is digested with a tobacco acid pyrophosphatase enzyme to give rise to the plurality of target messenger RNA molecules.

30. The method of claim 27, wherein the signaling means of each target messenger RNA molecule or fragment thereof comprises at least one signaling molecule attached to the messenger RNA molecule or fragment thereof, the signaling molecule selected from the group consisting of a fluorescent dye, biotin, and digoxigenin.

31. The method of claim 27, wherein each immobilized oligonucleotide has a free 3′ terminal hydroxyl group, and the orientation of each ligation template is such that the 5′ end of a specific target messenger RNA molecule or fragment thereof is ligated to a specific immobilized oligonucleotide.

32. The method of claim 27, wherein each immobilized oligonucleotide has a free 5′ terminal phosphate group and the orientation of each ligation template is such that the 3′ end of a specific target messenger RNA molecule or fragment thereof is ligated to a specific immobilized oligonucleotide.

33. The method of claim 8, wherein the population of nucleic acids that is analyzed is a population of cDNA molecules or genomic DNA molecules, and the plurality of target nucleic acids that is contacted with the array of immobilized oligonucleotides comprises a population of target DNA molecules corresponding to regions of interest in the cDNA molecules or genomic DNA molecules.

34. The method of claim 33, wherein the region of interest in a cDNA molecule is selected from the group consisting of a splice site, an alternative splice site, an alternative transcriptional start site, an alternative polyadenylation site, an edited region, and a polymorphic region.

35. The method of claim 33, wherein the region of interest in a genomic DNA molecule is selected from the group consisting of a single nucleotide polymorphism, a single point mutation, a methylated site, a transcription factor binding site, a small insertion, a small deletion, a small translocation, a single tandem repeat, and a small variable number of tandem repeats.

36. The method of claim 33, wherein the signaling means of each target DNA molecule comprises at least one signaling molecule attached to the DNA molecule, the signaling molecule selected from the group consisting of a fluorescent dye, biotin, digoxigenin, and a sequence of nucleotides that is a target for branched DNA detection.

37. The method of claim 33, wherein each immobilized oligonucleotide comprises a free 5′ terminal phosphate group, each target DNA molecule comprises a 5′ signaling molecule, and the orientation of each ligation template is such that the 3′ end of a specific target DNA molecule is ligated to a specific immobilized oligonucleotide.

38. A kit for analyzing at least one population of nucleic acids, the kit comprising:
(a) an array of immobilized oligonucleotides covalently attached to a solid support at a plurality of distinct array positions, each array position comprising at least one immobilized oligonucleotide comprising a unique artificial sequence;
(b) a plurality of ligation templates, each ligation template comprising a first region with complementarity to the unique artificial sequence of a specific immobilized oligonucleotide and a second region with complementarity to all or part of a specific target nucleic acid; and
(c) a template-dependent ligase.

39. The kit of claim 38, wherein the unique artificial sequence of each immobilized oligonucleotide is from about 4 nucleotides to about 30 nucleotides in length and is located at the free end of the immobilized oligonucleotide.

40. The kit of claim 38, wherein the solid support is a material selected from the group consisting of glasses, silicons, polymers, and metals; and the solid support has a form selected from the group consisting of a slide, a plate, a well, a microparticle, and a combination thereof.

41. The kit of claim 40, wherein the solid support is further modified to contain a thin layer of three dimensional porous structures selected from the group consisting of a hydrophilic polymer gel, a dendrimer, and a combination thereof.

42. The kit of claim 38, wherein the template-dependent ligase is selected from the group consisting of T4 DNA ligase, T4 RNA ligase 2, vaccinia DNA ligase, E. coli DNA ligase, a mammalian DNA ligase, Taq DNA ligase, Tth DNA ligase, Tfi DNA ligase, Ampligase DNA ligase, 9° N DNA ligase, and a combination thereof.

43. The kit of claim 38, wherein each ligation template further comprises at least one molecule selected from the group consisting of a locked nucleic acid, biotin, and digoxigenin.

44. The kit of claim 38, wherein each ligation template further comprises a region with complementarity to a portion of a detection tag.

44. The kit of claim 38, further comprising at least one detection tag, the detection tag comprising an oligonucleotide portion and at least one signaling molecule, the oligonucleotide portion for ligating the detection tag to the target nucleic acid, the signaling molecule selected from the group consisting of a fluorescent dye, biotin, digoxigenin, and a sequence of nucleotides that is a target for branched DNA detection.

45. The kit of claim 38, further comprising a signaling molecule for attachment to the target nucleic acid, the signaling molecule selected from the group consisting of a fluorescent dye, a luminescent dye, biotin, digoxigenin, and a sequence of nucleotides that is a target for branched DNA detection.
